(19) United States
(12) Patent Application Publication
(10) Pub. No.: US 2025/0179049 A1
(43) Pub. Date: Jun. 5, 2025

(54) SALTS OF HETEROCYCLIC INHIBITORS OF MONOCARBOXYLATE TRANSPORTER 4 FOR THE TREATMENT OF DISEASE

(71) Applicant: Vettore, LLC, San Francisco, CA (US)

(72) Inventors: Jon LAWSON, Wildwood, MO (US); Mark PARNELL, San Francisco, CA (US); Wei MA, San Francisco, CA (US); Peter VARLASHKIN, San Francisco, CA (US); Frank TARCZYNSKI, San Francisco, CA (US); Stephen CARINO, San Francisco, CA (US)

(21) Appl. No.: 19/047,401

(22) Filed: Feb. 6, 2025

Related U.S. Application Data
(63) Continuation of application No. PCT/US2023/071973, filed on Aug. 10, 2023.
(60) Provisional application No. 63/370,972, filed on Aug. 10, 2022.

(51) Int. Cl. C07D 403/10; A61K 31/4155; A61P 11/00

(52) U.S. Cl. CPC C07D 403/10; A61K 31/4155; A61P 11/00

(57) ABSTRACT

Disclosed herein are new salts of substituted pyrazole compounds which inhibit the activity of the monocarboxylate transporter MCT4, or a mutant thereof. Also disclosed herein are compounds, pharmaceutical compositions, and methods of treatment of MCT4-mediated diseases, such as inflammatory disorders and proliferative disorders.

This application is a bypass continuation of International Application No. PCT/US2023/071973, filed Aug. 10, 2023, which claims the benefit of priority of U.S. provisional application No. 63/370,972, filed Aug. 10, 2022, the contents of which are incorporated by reference as if written herein in their entireties.

Lactic acid export from glycolytic cells is typically mediated by the monocarboxylate transporter MCT4. MCT4 exhibits weak affinity for lactate (Kₘ=28 mM) coupled with a high turnover rate, allowing rapid export of large amounts of lactic acid. MCT4 expression is normally limited to highly glycolytic tissues such as white muscle fibers, lymphocytes, astrocytes, and Sertoli cells. Though MCT4 is absent from most normal tissues, MCT4 expression is highly upregulated, and correlates with poor survival, in many cancer indications, including colorectal cancer, glioma, head and neck cancer, triple-negative breast cancer, prostate cancer, KRAS mutant lung cancer, liver cancer, and kidney cancer.

The correlation of MCT4 expression and poor cancer outcome appears to be of significant functional consequence in multiple cancer models. The stable expression of MCT4 is highly tumorigenic in a respiration-impaired, Ras-transformed fibroblast xenograft model. Conversely, MCT4 silencing slows or ablates tumor growth in xenograft models of breast cancer, colorectal cancer, and glioma. MCT4 expression is required for inflammatory cytokine IL-8-mediated angiogenesis in breast and colon cancer xenograft models. MCT4 has also been shown to play important roles in cancer cell migration, invasion, and various aspects of the Warburg effect (e.g., proliferation on glucose, extracellular acidification, and lactate secretion).

Inhibition of MCT4-mediated lactic acid export may be an effective strategy to impair the Warburg effect in cancer. Unfortunately, no potent and selective MCT4 inhibitors have been described. Moderate to weak MCT4 inhibitors are known (e.g., phloretin and α-CN-4-OH-cinnamate); however, these compounds promiscuously inhibit a number of other transporters, including MCT1.

Novel salts and pharmaceutical compositions, which have been found to inhibit MCT4 have been discovered, together with methods of synthesizing and using the salts including methods for the treatment of MCT4-mediated diseases in a patient by administering the compounds.

### SUMMARY

Provided is Form A of a compound of structural Formula I

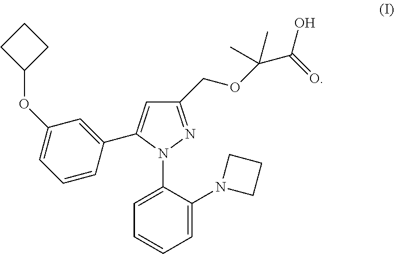 [SMILES: CC(C)(OCc1cc(-c2cccc(OC3CCC3)c2)n(-c2ccccc2N2CCC2)n1)C(=O)O]

Also provided is a compound of structural Formula I that is amorphous

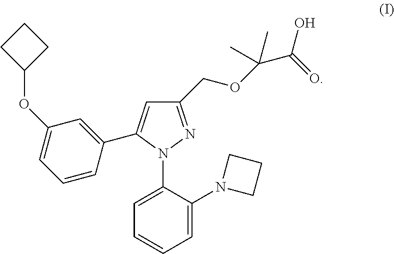 [SMILES: CC(C)(OCc1cc(-c2cccc(OC3CCC3)c2)n(-c2ccccc2N2CCC2)n1)C(=O)O]

Also provided is a compound of structural Formula II

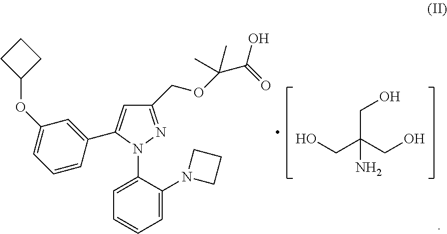

Also provided is Form A of a compound of structural Formula II

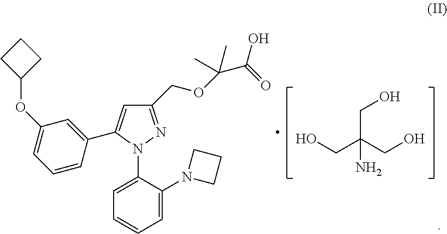

Also provided is Form B of a compound of structural Formula II

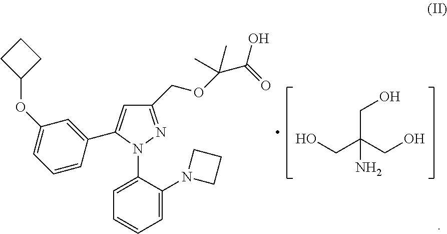

Also provided is a pharmaceutical composition comprising a compound described herein, and a pharmaceutically acceptable carrier, adjuvant, or vehicle.

Also provided is a method for inhibiting activity of the monocarboxylate transporter MCT4, or a mutant thereof, in a biological sample comprising the step of contacting the biological sample with a compound as described herein.

Also provided is a method for inhibiting activity of the monocarboxylate transporter MCT4, or a mutant thereof, in a patient comprising the step of administering to the patient a compound as described herein.

Also provided is a method for selectively inhibiting activity of the monocarboxylate transporter MCT4, or a mutant thereof, over the monocarboxylate transporter MCT1, or a mutant thereof, in a patient comprising the step of administering to the patient a compound as described herein.

Also provided is a method for treating a monocarboxylate transporter MCT4-mediated disorder in a subject in need thereof, comprising the step of administering to the patient a compound as described herein.

Also provided is a method of treating a monocarboxylate transporter MCT4-mediated disorder in a subject in need thereof, comprising the sequential or co-administration of a compound as described herein, and another therapeutic agent.

Also provided is a method for achieving an effect in a patient comprising the administration of a therapeutically effective amount of a compound as described herein to a patient, wherein the effect is selected from the group consisting of reduction of triglycerides, reduction of cholesterol, and reduction of hemoglobin A1c.

These and other aspects of the invention will be apparent upon reference to the following description. To this end, various references are set forth herein which describe in more detail certain background information, procedures, compounds, and/or compositions, and are each hereby incorporated by reference in their entirety.

### BRIEF DESCRIPTION OF THE DRAWINGS

FIG. 1 shows the FT-Raman spectrum of a compound of structural Formula I.

FIG. 2 shows the DSC and TGA traces of a compound of structural Formula I.

FIG. 3 shows the XRPD diffractogram of a compound of structural Formula I.

FIG. 4 shows the ¹H-NMR spectrum of a compound of structural Formula I.

FIG. 5 shows the FT-Raman spectrum of Form A of a compound of structural Formula II.

FIG. 6 shows the DSC and TGA traces of Form A of a compound of structural Formula II.

FIG. 7 shows the XRPD diffractogram of Form A of a compound of structural Formula II.

FIG. 8 shows the ¹H-NMR spectrum of Form A of a compound of structural Formula II.

FIG. 9 shows the Dynamic Vapor Sorption plot of the Form B of a compound of structural Formula II.

FIG. 10 shows the DSC and TGA traces of the Form B of a compound of structural Formula II.

FIG. 11 shows the XRPD diffractogram of the Form B of a compound of structural Formula II.

FIG. 12 shows the ¹H-NMR spectrum of the Form B of a compound of structural Formula II.

FIG. 13 shows mean plasma concentrations after oral dosing of a compound of structural Formula I in male rats at 20, 60, and 200 mg/kg.

FIG. 14 shows mean plasma concentrations after oral dosing of a compound of structural Formula I in female rats at 20, 60, and 200 mg/kg.

FIG. 15 shows mean plasma concentrations after oral dosing of a compound of structural Formula II in male rats at 25, 75, and 250 mg/kg.

FIG. 16 shows mean plasma concentrations after oral dosing of a compound of structural Formula II in female rats at 25, 75, and 250 mg/kg.

FIG. 17 shows mean plasma concentrations after oral dosing of a compound of structural Formula I in male dogs at 3 mg/kg.

FIG. 18 shows mean plasma concentrations after oral dosing of a compound of structural Formula II in male dogs at 3 mg/kg.

FIG. 19 shows plasma concentrations after oral dosing of a compound of structural Formula II in male dogs at 3 mg/kg.

FIG. 20 shows plasma concentrations after oral dosing of a compound of structural Formula II in male dogs at 30 mg/kg.

FIG. 21 shows a schematic of potential dose groups for Parts A and C of the Phase 1 protocol.

FIG. 22 shows a schematic of potential dose groups for Parts B and D of the Phase 1 protocol.

FIG. 23 shows the results of a bleomycin-induced lung fibrosis model in mice. Compound 1 (VB253) was dosed at 3 mg/kg, BID; pirfenidone was dosed at 100 mg/kg, BID; and nintedanib was dosed at 50 mg/kg, QD, all via oral gavage.

### DETAILED DESCRIPTION

Provided is Form A of a compound of structural Formula I

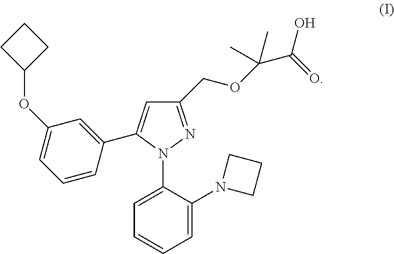 [SMILES: CC(C)(OCc1cc(-c2cccc(OC3CCC3)c2)n(-c2ccccc2N2CCC2)n1)C(=O)O]

The compound of structural Formula I is also referred to herein as Compound 1 or 2-((1-(2-(azetidin-1-yl)phenyl)-5-(3-cyclobutoxyphenyl)-1H-pyrazol-3-yl) methoxy)-2-methylpropanoic acid. In certain embodiments, Compound 1 is administered as a pharmaceutically acceptable salt, such as the tris salt of 2-((1-(2-(azetidin-1-yl)phenyl)-5-(3-cyclobutoxyphenyl)-1H-pyrazol-3-yl) methoxy)-2-methylpropanoic acid.

In certain embodiments, Form A of a compound of structural Formula I is non-solvated.

In certain embodiments, Form A of a compound of structural Formula I has differential scanning calorimetry data showing a primary melting endotherm with onset at about 157° C.

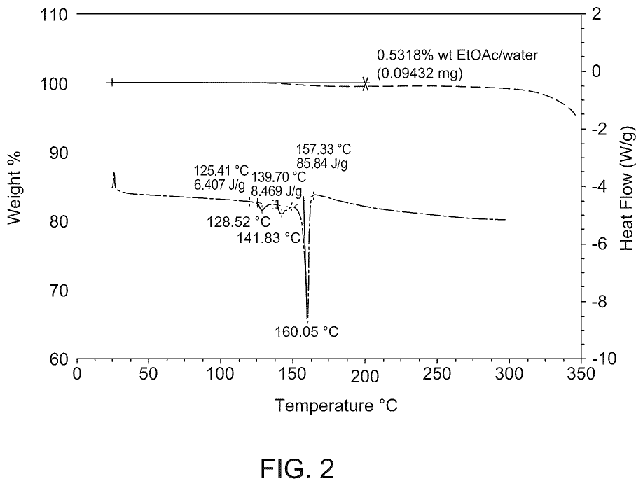

In certain embodiments, Form A of a compound of structural Formula I has a differential scanning calorimetry trace substantially as shown in FIG. 2.

In certain embodiments, Form A of a compound of structural Formula I has a TGA trace substantially as shown in FIG. 2.

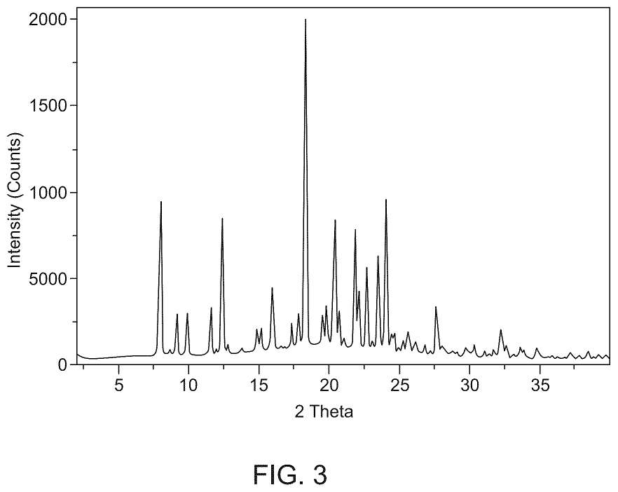

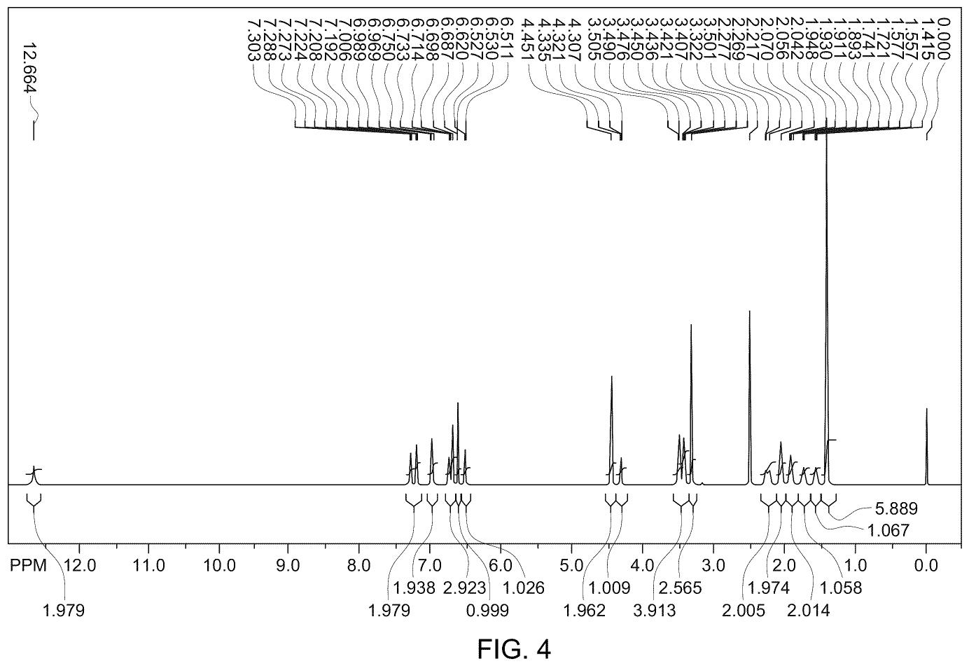

In certain embodiments, Form A of a compound of structural Formula I has an X-ray powder diffraction (XRPD) pattern substantially as shown in FIG. 3.

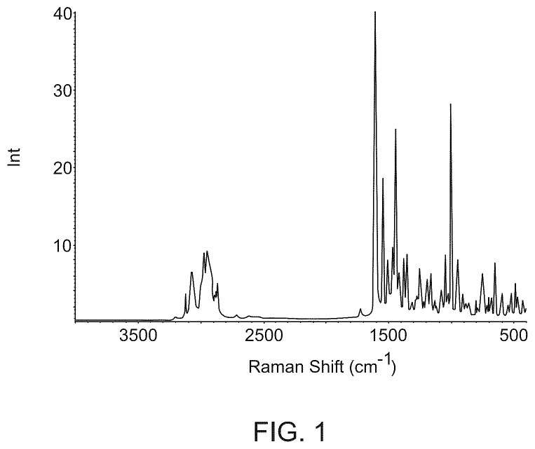

In certain embodiments, Form A of a compound of structural Formula I has an FT-Raman spectrum substantially as shown in FIG. 1.

Also provided is Form A of a compound of structural Formula I prepared by a process described herein.

Also provided is a compound of structural Formula I that is amorphous

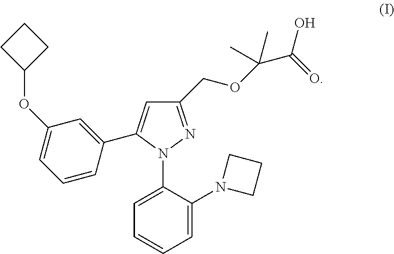 [SMILES: CC(C)(OCc1cc(-c2cccc(OC3CCC3)c2)n(-c2ccccc2N2CCC2)n1)C(=O)O]

Also provided is a compound of structural Formula II

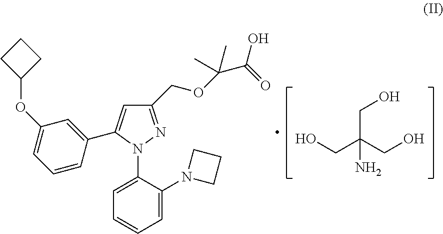

The compound of structural Formula II is also referred to herein as the tris salt or Compound 1 tris salt.

In certain embodiments, the compound of structural Formula II has a stoichiometry of active ingredient to counterion of 1:1.

Also provided is Form A of a compound of structural Formula II

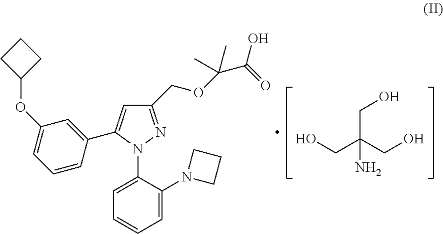

In certain embodiments, Form A of a compound of structural Formula II is non-solvated.

In certain embodiments, Form A of a compound of structural Formula II has a stoichiometry of active ingredient to counterion of 1:1.

In certain embodiments, Form A of a compound of structural Formula II has an X-ray powder diffraction (XRPD) pattern with peaks at about 7.79, 15.61, 16.71, 20.00, and 20.88±0.3 degrees two theta wherein the XRPD is measured using an incident beam of Cu radiation.

In certain embodiments, Form A of a compound of structural Formula II has an X-ray powder diffraction (XRPD) pattern with peaks at about 7.79, 12.59, 15.61, 16.71, 20.00, 20.88, and 21.50±0.3 degrees two theta wherein the XRPD is measured using an incident beam of Cu radiation.

In certain embodiments, Form A of a compound of structural Formula II has an X-ray powder diffraction (XRPD) pattern with peaks at about 7.79, 12.18, 12.59, 15.61, 16.71, 17.38, 17.72, 19.16, 20.00, 20.88, and 21.50±0.3 degrees two theta wherein the XRPD is measured using an incident beam of Cu radiation.

In certain embodiments, Form A of a compound of structural Formula II has an X-ray powder diffraction (XRPD) pattern with peaks at about 11.34, 5.67, 5.30, 4.44, and 4.25±0.3 Å in d-spacing, wherein the XRPD is measured using an incident beam of Cu radiation.

In certain embodiments, Form A of a compound of structural Formula II has an X-ray powder diffraction (XRPD) pattern with peaks at about 11.34, 7.02, 5.67, 5.30, 4.44, 4.25 and 4.13±0.3 Å in d-spacing, wherein the XRPD is measured using an incident beam of Cu radiation.

In certain embodiments, Form A of a compound of structural Formula II has an X-ray powder diffraction (XRPD) pattern with peaks at about 11.34, 7.26, 7.02, 5.67, 5.30, 5.10, 5.00, 4.63, 4.44, 4.25, and 4.13 Å in d-spacing, wherein the XRPD is measured using an incident beam of Cu radiation.

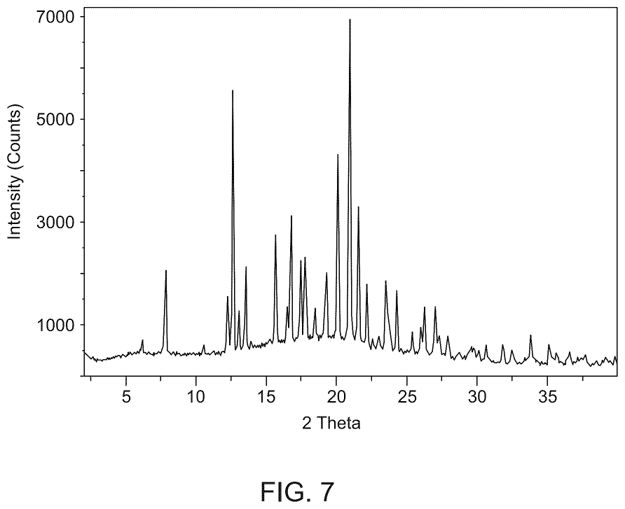

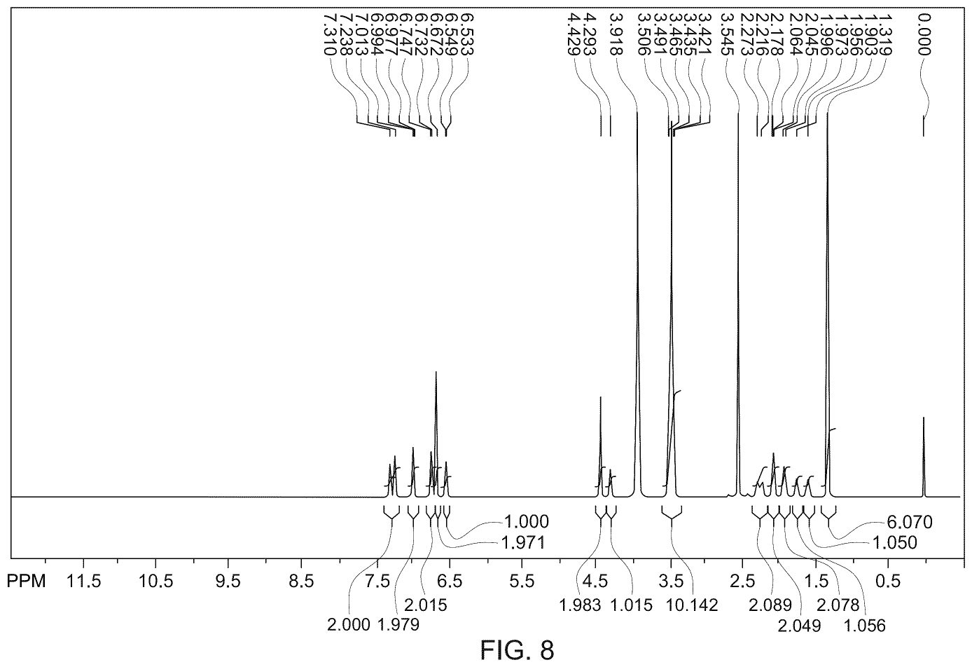

In certain embodiments, Form A of a compound of structural Formula II has an X-ray powder diffraction (XRPD) pattern substantially as shown in FIG. 7.

In certain embodiments, Form A of a compound of structural Formula II is characterized by the presence of FT-Raman peaks of about 1604, 1438, and 995 cm⁻¹.

In certain embodiments, Form A of a compound of structural Formula II is characterized by the presence of FT-Raman peaks of about 1604, 1438, 1372, 995, 332, 234, and 173 cm⁻¹.

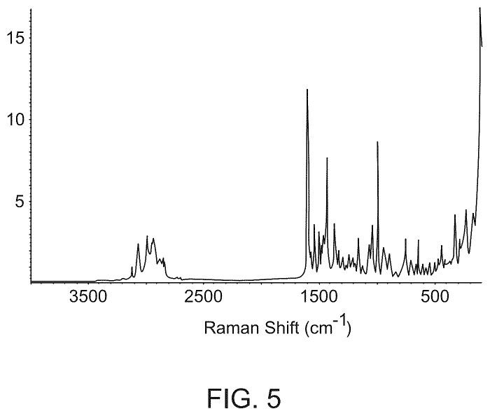

In certain embodiments, Form A of a compound of structural Formula II has an FT-Raman spectrum substantially as shown in FIG. 5.

In some embodiments, the compound is characterized by a weight loss of no more than 0.5% between about 25° C. and 150° C. by thermogravimetric analysis (TGA).

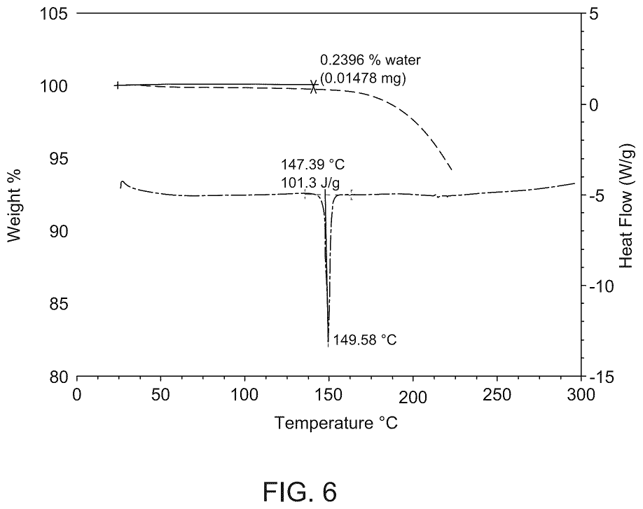

In some embodiments, Form A of a compound of structural Formula II has a TGA trace substantially as shown in FIG. 6.

In certain embodiments, Form A of a compound of structural Formula II has differential scanning calorimetry data showing a melting endotherm with onset at about 148° C.

In certain embodiments, Form A of a compound of structural Formula II has a differential scanning calorimetry trace substantially as shown in FIG. 6.

Also provided is a process for making Form A of a compound of structural Formula II comprising combining a compound of structural Formula I with tris(hydroxymethyl)aminomethane, in a solvent and isolating Form A of the compound of structural Formula II. In some embodiments, the solvent is chosen from water, acetone, and acetonitrile, or a mixture thereof. In some embodiments, the combining occurs at room temperature.

Also provided is Form A of a compound of structural Formula II prepared by a process described herein.

Also provided is Form B of a compound of structural Formula II

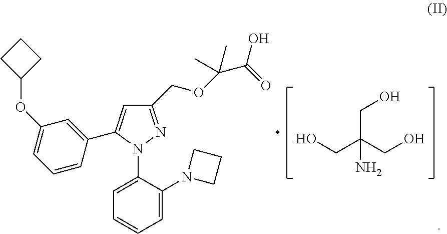

In certain embodiments, Form B of a compound of structural Formula II is non-solvated.

In certain embodiments, Form B of a compound of structural Formula II has a stoichiometry of active ingredient to counterion of 1:1.

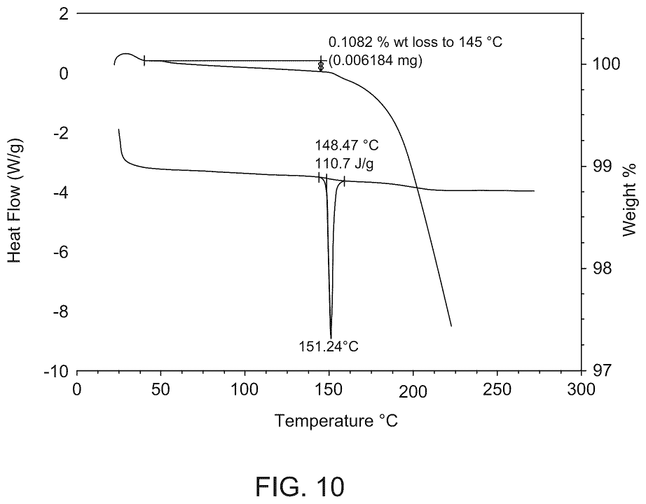

In certain embodiments, Form B of a compound of structural Formula II has a differential scanning calorimetry trace substantially as shown in FIG. 10.

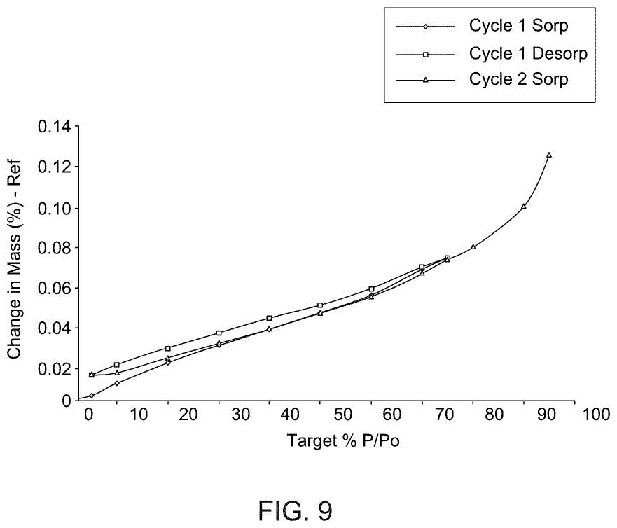

In certain embodiments, Form B of a compound of structural Formula II has a Dynamic Vapor Sorption plot substantially as shown in FIG. 9.

In certain embodiments, Form B of a compound of structural Formula II has an X-ray powder diffraction (XRPD) pattern with peaks at about 9.29, 9.70, 16.36, 19.12, and 20.15 degrees two theta wherein the XRPD is measured using an incident beam of Cu radiation.

In certain embodiments, Form B of a compound of structural Formula II has an X-ray powder diffraction (XRPD) pattern with peaks at about 9.29, 9.70, 10.03, 16.36, 19.12, 19.49, 19.61, 20.15, and 21.68 degrees two theta wherein the XRPD is measured using an incident beam of Cu radiation.

In certain embodiments, Form B of a compound of structural Formula II has an X-ray powder diffraction (XRPD) pattern with peaks at about 9.29, 9.70, 10.03, 11.14, 11.73, 16.36, 16.71, 19.12, 19.49, 19.61, 20.15, 20.52, 20.73, and 21.68 degrees two theta wherein the XRPD is measured using an incident beam of Cu radiation.

In certain embodiments, Form B of a compound of structural Formula II has an X-ray powder diffraction (XRPD) pattern with peaks at about 9.51, 9.11, 5.41, 4.64, and 4.40 Å in d-spacing, wherein the XRPD is measured using an incident beam of Cu radiation.

In certain embodiments, Form B of a compound of structural Formula II has an X-ray powder diffraction (XRPD) pattern with peaks at about 9.51, 9.11, 8.81, 5.41, 4.64, 4.55, 4.52, 4.40, and 4.10 Å in d-spacing, wherein the XRPD is measured using an incident beam of Cu radiation.

In certain embodiments, Form B of a compound of structural Formula II has an X-ray powder diffraction (XRPD) pattern with peaks at about 9.51, 9.11, 8.81, 7.93, 7.54, 5.41, 5.30, 4.64, 4.55, 4.52, 4.40, 4.32, 4.28 and 4.10 Å in d-spacing, wherein the XRPD is measured using an incident beam of Cu radiation.

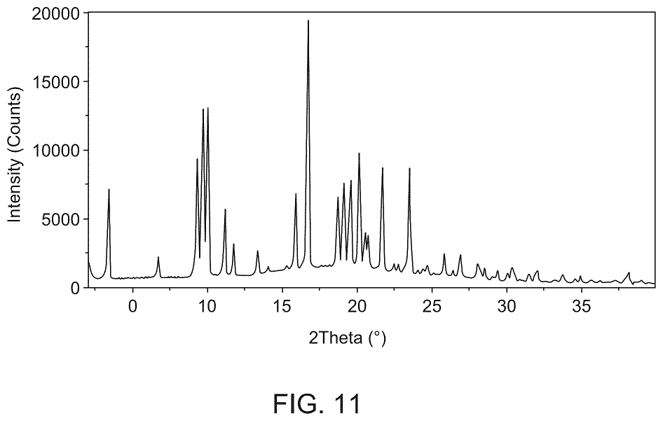

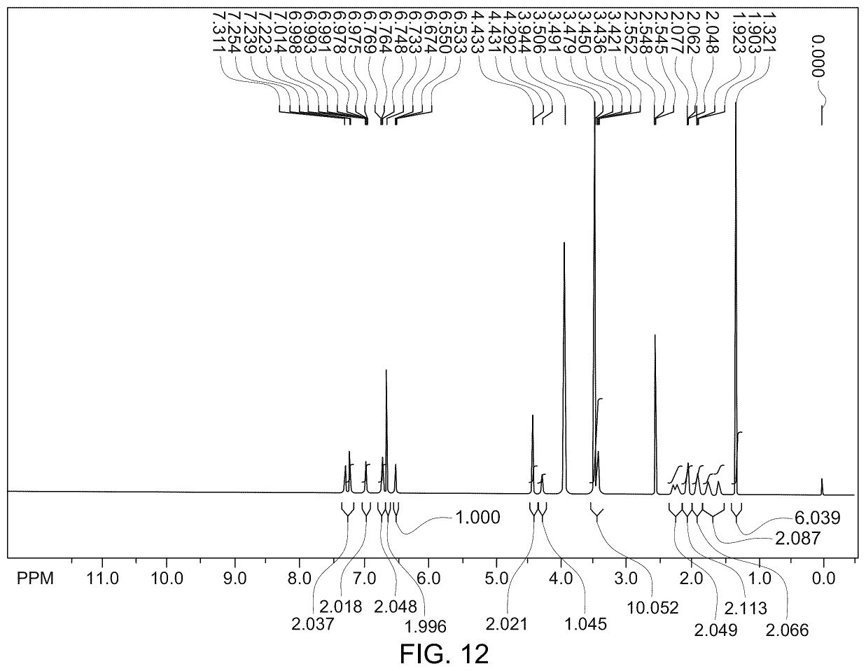

In certain embodiments, Form B of a compound of structural Formula II has an X-ray powder diffraction (XRPD) pattern substantially as shown in FIG. 11.

In certain embodiments, Form B of a compound of structural Formula II is characterized by a monoclinic lattice type and P2₁/c space group having unit cell lengths for the three axes of about (a) 26.526 A, (b) 5.940 A, (c) 19.055 A and the three unit cell angles of about (a) 90.00°, (β) 90.00°, and (γ) 93.123°.

In certain embodiments, Form B of a compound of structural Formula II is characterized by the presence of FT-Raman peaks of about 1601, 1545, 1468, 1437, 999, 995, and 234 cm⁻¹.

In certain embodiments, Form B of a compound of structural Formula II is characterized by the presence of FT-Raman peaks of about 2946, 1601, 1545, 1507, 1468, 1437, 1374, 1345, 1043, 999, 995, 284, 234, and 186 cm⁻¹.

In certain embodiments, Form B of a compound of structural Formula II has differential scanning calorimetry data showing a melting endotherm with onset at about 148° C.

In some embodiments, the compound is characterized by a weight loss of no more than 0.1% between about 25° C. and 145° C. by thermogravimetric analysis (TGA).

In some embodiments, Form B of a compound of structural Formula II has a TGA trace substantially as shown in FIG. 10.

Also provided is a process for making Form B of a compound of structural Formula II, comprising stirring Form A of a compound of structural Formula II with a suitable solvent and adding a seed crystal of Form B of a compound of structural Formula II, and isolating Form B of a compound of structural Formula II. Also provided is Form B of a compound of structural Formula II prepared by the processes described herein.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Certain compounds disclosed herein may possess useful MCT4 inhibiting activity and may be used in the treatment or prophylaxis of a disease or condition in which MCT4 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting MCT4. Other embodiments provide methods for treating a MCT4-mediated disorder in a patient in need of such treatment, comprising administering to the patient a therapeutically effective amount of a compound or composition according to the present invention. Also provided is the use of certain compounds disclosed herein for use in the manufacture of a medicament for the treatment of a disease or condition ameliorated by the inhibition of MCT4.

Provided is a method of inhibiting at least one MCT4 function comprising the step of contacting MCT4 with a compound as described herein. The cell phenotype, cell proliferation, activity of MCT4, change in biochemical output produced by active MCT4, expression of MCT4, or binding of MCT4 with a natural binding partner may be monitored. Such methods may be modes of treatment of disease, biological assays, cellular assays, biochemical assays, or the like.

Also provided herein is a method of treatment of a MCT4-mediated disease comprising the administration of a therapeutically effective amount of a compound as disclosed herein, to a patient in need thereof.

In certain embodiments, the disease is chosen from proliferative inflammatory diseases.

In certain embodiments, the disease is a metabolic disease.

In certain embodiments, the metabolic disease is chosen from metabolic syndrome, diabetes, dyslipidemia, fatty liver disease, non-alcoholic steatohepatitis, obesity, and insulin resistance.

In certain embodiments, the diabetes is Type II diabetes.

In certain embodiments, the dyslipidemia is hyperlipidemia.

Further provided is a method for achieving an effect in a patient comprising the administration of a therapeutically effective amount of a compound as disclosed above to a patient, wherein the effect is selected from the group consisting of reduction of triglycerides, reduction of cholesterol, and reduction of hemoglobin A1c.

Further provided is the method as disclosed above wherein the cholesterol is chosen from LDL and VLDL cholesterol.

Further provided is the method as disclosed above wherein the triglycerides are chosen from plasma triglycerides and liver triglycerides.

Also provided herein is a method of inhibition of MCT4 comprising contacting MCT4 with a compound as disclosed herein.

Also provided herein is a method for achieving an effect in a patient comprising the administration of a therapeutically effective amount of a compound as disclosed herein, to a patient, wherein the effect is chosen from cognition enhancement.

In certain embodiments, the MCT4-mediated disease is chosen from proliferative inflammatory diseases.

Also provided is a method of modulation of a MCT4-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the oral pharmaceutical composition is chosen from a tablet and a capsule.

### Abbreviations and Definitions

As used herein, the terms below have the meanings indicated.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “MCT4 inhibitor” is used herein to refer to a compound that exhibits an IC₅₀ with respect to MCT4 activity of no more than about 100 μM and more typically not more than about 50 μM, as measured in the MCT4 enzyme assay described generally herein below. IC₅₀ is that concentration of inhibitor that reduces the activity of an enzyme (e.g., MCT4) to half-maximal level. Certain compounds disclosed herein have been discovered to exhibit inhibition against MCT4. In certain embodiments, compounds will exhibit an IC₅₀ with respect to MCT4 of no more than about 10 μM; in further embodiments, compounds will exhibit an IC₅₀ with respect to MCT4 of no more than about 5 μM; in yet further embodiments, compounds will exhibit an IC₅₀ with respect to MCT4 of not more than about 1 μM; in yet further embodiments, compounds will exhibit an IC₅₀ with respect to MCT4 of not more than about 200 nM, as measured in the MCT4 binding assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g., orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Thus, in another aspect, certain embodiments provide methods for treating MCT4-mediated disorders in a human or animal subject in need of such treatment comprising administering to the subject an amount of a compound disclosed herein effective to reduce or prevent the disorder in the subject, in combination with at least one additional agent for the treatment of the disorder that is known in the art. In a related aspect, certain embodiments provide therapeutic compositions comprising at least one compound disclosed herein in combination with one or more additional agents for the treatment of MCT4-mediated disorders.

Also provided herein is a method of treating a monocarboxylate transporter MCT4-mediated disorder in a subject in need thereof, comprising the sequential or co-administration of a compound as disclosed herein, and another therapeutic agent.

In certain embodiments, the therapeutic agent is a protein kinase inhibitor.

In certain embodiments, the protein kinase inhibitor is chosen from Aurora B, EGFR, PLK-1, CDKs inhibitors.

In certain embodiments, the therapeutic agent is chosen from an antimetabolite, bcr-abl inhibitor, DNA damaging agent, EGFR inhibitor, microtubule stabilizing inhibitor, mitotic arrest inhibitor, S-phase inhibitor, and a taxane.

In certain embodiments, the therapeutic agent is a DNA damaging agent chosen from an alkylating agent, anthracycline, antimetabolite agent, crosslinking agent, DNA replication inhibitor, intercalator, microtubule disruptor, PARP inhibitor, radiomimetic agent, radiosensitizer, strand break agent, and topoisomerase II inhibitor.

In certain embodiments, the therapeutic agent is chosen from aminoglutethimide, amsacrine, anastrozole, asparaginase, barasertib, bcg, bicalutamide, bleomycin, buserelin, busulfan, campothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, olaparib, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, and vinorelbine.

In certain embodiments, the therapeutic agent is chosen from paracetamol, acetaminophen, pirfenidone, nintedanib, and non-hormonal contraceptives.

For use in cancer and neoplastic diseases a MCT4 inhibitor may be optimally used together with one or more of the following non-limiting examples of anti-cancer agents: (1) alkylating agents, including but not limited to cisplatin (PLATIN), carboplatin (PARAPLATIN), oxaliplatin (ELOXATIN), streptozocin (ZANOSAR), busulfan (MYLERAN) and cyclophosphamide (ENDOXAN); (2) anti-metabolites, including but not limited to mercaptopurine (PURINETHOL), thioguanine, pentostatin (NIPENT), cytosine arabinoside (ARA-C), gemcitabine (GEMZAR), fluorouracil (CARAC), leucovorin (FUSILEV) and methotrexate (RHEUMATREX); (3) plant alkaloids and terpenoids, including but not limited to vincristine (ONCOVIN), vinblastine and paclitaxel (TAXOL); (4) topoisomerase inhibitors, including but not limited to irinotecan (CAMPTOSAR), topotecan (HYCAMTIN) and etoposide (EPOSIN); (5) cytotoxic antibiotics, including but not limited to actinomycin D (COSMEGEN), doxorubicin (ADRIAMYCIN), bleomycin (BLENOXANE) and mitomycin (MITOSOL); (6) angiogenesis inhibitors, including but not limited to sunitinib (SUTENT) and bevacizumab (AVASTIN); and (7) tyrosine kinase inhibitors, including but not limited to imatinib (GLEEVEC), erlotinib (TARCEVA), lapatininb (TYKERB) and axitinib (INLYTA).

Where a subject is suffering from or at risk of suffering from an inflammatory condition, a MCT4 inhibitor compound described herein is optionally used together with one or more agents or methods for treating an inflammatory condition in any combination. Therapeutic agents/treatments for treating an autoimmune and/or inflammatory condition include, but are not limited to any of the following examples: (1) corticosteroids, including but not limited to cortisone, dexamethasone, and methylprednisolone; (2) nonsteroidal anti-inflammatory drugs (NSAIDs), including but not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN) and piroxicam (FELDENE); (3) immunosuppressants, including but not limited to methotrexate (RHEUMATREX), leflunomide (ARAVA), azathioprine (IMURAN), cyclosporine (NEORAL, SANDIMMUNE), tacrolimus and cyclophosphamide (CYTOXAN); (4) CD₂₀ blockers, including but not limited to rituximab (RITUXAN); (5) Tumor Necrosis Factor (TNF) blockers, including but not limited to etanercept (ENBREL), infliximab (REMICADE) and adalimumab (HUMIRA); (6) interleukin-1 receptor antagonists, including but not limited to anakinra (KINERET); (7) interleukin-6 inhibitors, including but not limited to tocilizumab (ACTEMRA); (8) interleukin-17 inhibitors, including but not limited to AIN₄₅₇; (9) Janus kinase inhibitors, including but not limited to tasocitinib; and (10) syk inhibitors, including but not limited to fostamatinib.

In certain embodiments, the method further comprises administering non-chemical methods of cancer treatment.

In certain embodiments, the method further comprises administering radiation therapy.

In certain embodiments, the method further comprises administering surgery, thermoablation, focused ultrasound therapy, cryotherapy, or any combination thereof.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Also provided are methods for treating MCT4-mediated disorders in a human or animal subject in need of such treatment comprising administering to the subject an amount of a compound disclosed herein effective to reduce or prevent the disorder in the subject, in combination with at least one additional agent for the treatment of the disorder that is known in the art. In a related aspect, certain embodiments provide therapeutic compositions comprising at least one compound disclosed herein in combination with one or more additional agents for the treatment of MCT4-mediated disorders.

Also provided are compounds and pharmaceutical compositions that inhibit glutaminase activity, particularly MCT4 activity and are thus useful in the treatment or prevention of disorders associated with MCT4. Compounds and pharmaceutical compositions of described herein selectively modulate MCT4 and are thus useful in the treatment or prevention of a range of disorders associated with MCT4 and include, but are not limited to, proliferative and inflammatory diseases.

Accordingly, provided herein is a method for inhibiting activity of the monocarboxylate transporter MCT4, or a mutant thereof, in a biological sample comprising the step of contacting the biological sample with a compound as disclosed herein.

Also provided herein is a method for inhibiting activity of the monocarboxylate transporter MCT4, or a mutant thereof, in a patient comprising the step of administering to the patient a compound as disclosed herein.

Also provided herein is a method for selectively inhibiting activity of the monocarboxylate transporter MCT4, or a mutant thereof, over the monocarboxylate transporter MCT1, or a mutant thereof, in a patient comprising the step of administering to the patient a compound as disclosed herein.

In certain embodiments, the inhibition is at least 100-fold selective for MCT4 over MCT1.

In certain embodiments, the compounds, salts, and pharmaceutical compositions described herein may be useful in the treatment or prevention of cancer.

In certain embodiments, the compounds and salts described herein may be used to prevent or treat cancer, wherein the cancer is one or a variant of Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sézary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sézary Syndrome), Hodgkin, Non-Hodgkin, Primary Central Nervous System (CNS), Macroglobulinemia, Waldenström, Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (like Ewing Sarcoma Family of Tumors, Kaposi, Soft Tissue, Uterine), Sézary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, Supratentorial Primitive Neuroectodermal Tumors, T-Cell Lymphoma (Cutaneous, Mycosis Fungoides and Sézary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenström Macroglobulinemia or Wilms Tumor.

In certain embodiments, the cancer to be treated is one specific to T-cells such as T-cell lymphoma and lymphoblastic T-cell leukemia.

In certain embodiments, methods described herein are used to treat a disease condition comprising administering to a subject in need thereof a therapeutically effective amount of a compound as described herein, wherein the condition is cancer which has developed resistance to chemotherapeutic drugs and/or ionizing radiation.

In certain embodiments, the compounds, salts, and pharmaceutical compositions described herein are useful in the treatment or prevention of an inflammatory disease.

In certain embodiments, the compounds and salts described herein may be used to prevent or treat inflammatory disease, wherein the inflammatory disease is one or a variant of acid-induced lung injury, acne (PAPA), acute respiratory distress syndrome, Addison's disease, adrenal hyperplasia, adrenocortical insufficiency, ageing, AIDS, alcoholic hepatitis, alcoholic liver disease, allergen induced asthma, allergic bronchopulmonary aspergillosis, allergic conjunctivitis, alopecia, Alzheimer's disease, amyloidosis, amyotrophic lateral sclerosis, angina pectoris, angioedema, anhidrotic ectodermal dysplasia (e.g. with immune deficiency), ankylosing spondylitis, anterior segment inflammation, antiphospholipid syndrome, aphthous stomatitis, appendicitis, asthma, atherosclerosis, atopic dermatitis, autoimmune diseases, autoimmune hepatitis, bee sting-induced inflammation, Behcet's disease, Bell's Palsy, berylliosis, Blau syndrome, bone pain, bronchiolitis, burns, bursitis, cardiac hypertrophy, carpal tunnel syndrome, catabolic disorders, cataracts, cerebral aneurysm, chemical irritant-induced inflammation, chorioretinitis, chronic heart failure, chronic lung disease of prematurity, chronic obstructive pulmonary disease, colitis, complex regional pain syndrome, connective tissue disease, corneal ulcer, Crohn's disease, cryopyrin-associated periodic syndromes, cryptococcosis, cystic fibrosis, deficiency of the interleukin-1-receptor antagonist, dermatitis, dermatitis endotoxemia, dermatomyositis, endometriosis, endotoxemia, epicondylitis, erythroblastopenia, familial amyloidotic polyneuropathy, familial cold urticaria, familial Mediterranean fever, fetal growth retardation, glaucoma, glomerular disease, glomerular nephritis, gout, gouty arthritis, graft-versus-host disease, gut diseases, head injury, headache, hearing loss, heart disease, hemolytic anemia, Henoch-Scholein purpura, hepatitis, hereditary periodic fever syndrome, herpes zoster and simplex, HIV-1, Huntington's disease, hyaline membrane disease, hyperammonemia, hypercalcemia, hypercholesterolemia, hyperimmunoglobulinemia D with recurrent fever, hypoplastic and other anemias, idiopathic pulmonary fibrosis, idiopathic thrombocytopenia purpura, incontinentia pigmenti, infectious mononucleosis, inflammatory bowel disease, inflammatory lung disease, inflammatory neuropathy, inflammatory pain, insect bite-induced inflammation, iritis, ischemia/reperfusion, juvenile rheumatoid arthritis, keratitis, kidney disease, kidney injury caused by parasitic infections, kidney transplant rejection prophylaxis, leptospirosis, Loeffler's syndrome, lung injury, lupus, lupus nephritis, meningitis, mesothelioma, mixed connective tissue disease, Muckle-Wells syndrome (urticaria deafness amyloidosis), multiple sclerosis, muscle wasting, muscular dystrophy, myasthenia gravis, myocarditis, mycosis fungoides, myelodysplastic syndrome, myositis, nasal sinusitis, necrotizing enterocolitis, neonatal onset multisystem inflammatory disease (NOMID), nephrotic syndrome, neuritis, neuropathological diseases, non-allergen induced asthma, obesity, ocular allergy, optic neuritis, organ transplant, osteoarthritis, otitis media, Paget's disease, pain, pancreatitis, Parkinson's disease, pemphigus, pericarditis, periodic fever, periodontitis, pertussis, perineal or peritoneal endometriosis, pharyngitis and adenitis (PFAPA syndrome), plant irritant-induced inflammation, pneumocystis infection, pneumonia, pneumonitis, poison ivy/urushiol oil-induced inflammation, polyarteritis nodosa, polychondritis, polycystic kidney disease, polymyositis, psoriasis, psychosocial stress disease, pulmonary disease, pulmonary fibrosis, pulmonary hypertension, pyoderma gangrenosum, pyogenic sterile arthritis, renal disease, retinal disease, rheumatic disease, rheumatoid arthritis, rheumatic carditis, sarcoidosis, sebborrhea, sepsis, severe pain, sickle cell, sickle cell anemia, silica-induced diseases, Sjogren's syndrome, skin diseases, sleep apnea, spinal cord injury, Stevens-Johnson syndrome, stroke, subarachnoid hemorrhage, sunburn, systemic sclerosis (scleroderma), temporal arteritis, tenosynovitis, thrombocytopenia, thyroiditis, tissue transplant, TNF receptor associated periodic syndrome (TRAPS), Toxoplasmosis, transplant, traumatic brain injury, tuberculosis, type 1 diabetes, type 2 diabetes, ulcerative colitis, urticaria, uveitis, Wegener's granulomatosis, and weight loss.

Thus, in another aspect, certain embodiments provide methods for treating a monocarboxylate transporter MCT4-mediated disorder in a subject in need thereof, comprising the step of administering to the patient a therapeutically effective amount of a compound as disclosed herein.

In certain embodiments, the subject is a human.

In certain embodiments, the subject is in a fed state.

In certain embodiments, the subject is in a fasted state.

In certain embodiments, the monocarboxylate transporter MCT4-mediated disorder is chosen from an inflammatory disorder and a proliferative disorder.

In certain embodiments, the monocarboxylate transporter MCT4-mediated disorder is a proliferative disorder.

In certain embodiments, the proliferative disorder is cancer.

In certain embodiments, the cancer is chosen from adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, and Wilms' tumor.

In certain embodiments, the monocarboxylate transporter MCT4-mediated disorder is an inflammatory disorder.

In certain embodiments, the inflammatory disorder is chosen from Crohn's disease, ulcerative colitis, idiopathic pulmonary fibrosis, muscular dystrophy, rheumatoid arthritis, and systemic sclerosis (scleroderma). In certain embodiments, the inflammatory disorder is idiopathic pulmonary fibrosis.

In certain embodiments, the therapeutically effective amount is between about 30 mg and about 200 mg. In certain embodiments, the therapeutically effective amount is between about 30 mg and about 80 mg. In certain embodiments, the therapeutically effective amount is chosen from 50 mg, 75 mg, 100 mg, 150 mg, and 200 mg.

Also provided herein is a compound as disclosed herein, for use in human therapy.

Also provided herein is a compound as disclosed herein, for use in treating a monocarboxylate transporter MCT4-mediated disorder, for example as disclosed in any of the embodiments and paragraphs above pertaining to methods of treatment.

Also provided herein is the use of a compound as disclosed herein for the manufacture of a medicament to treat a monocarboxylate transporter MCT4-mediated disorder, for example as disclosed in any of the embodiments and paragraphs above pertaining to methods of treatment.

Metabolic syndrome (also known as metabolic syndrome X) is characterized by having at least three of the following symptoms: insulin resistance; abdominal fat—in men this is defined as a 40 inch waist or larger, in women 35 inches or larger; high blood sugar levels—at least 110 milligrams per deciliter (mg/dL) after fasting; high triglycerides—at least 150 mg/dL in the blood stream; low HDL—less than 40 mg/dL; pro-thrombotic state (e.g. high fibrinogen or plasminogen activator inhibitor in the blood); or blood pressure of 130/85 mmHg or higher. A connection has been found between metabolic syndrome and other conditions such as obesity, high blood pressure and high levels of LDL cholesterol, all of which are risk factors for cardiovascular diseases. For example, an increased link between metabolic syndrome and atherosclerosis has been shown. People with metabolic syndrome are also more prone to developing type 2 diabetes, as well as PCOS (polycystic ovarian syndrome) in women and prostate cancer in men.

As described above, insulin resistance can be manifested in several ways, including type 2 diabetes. Type 2 diabetes is the condition most obviously linked to insulin resistance. Compensatory hyperinsulinemia helps maintain normal glucose levels, often for decades, before overt diabetes develops. Eventually the beta cells of the pancreas are unable to overcome insulin resistance through hypersecretion. Glucose levels rise, and a diagnosis of diabetes can be made. Patients with type 2 diabetes remain hyperinsulinemic until they are in an advanced stage of disease. As described above, insulin resistance can also correlate with hypertension. One half of patients with essential hypertension are insulin resistant and hyperinsulinemic, and there is evidence that blood pressure is linked to the degree of insulin resistance. Hyperlipidemia, too, is associated with insulin resistance. The lipid profile of patients with type 2 diabetes includes increased serum very-low-density lipoprotein cholesterol and triglyceride levels and, sometimes, a decreased low-density lipoprotein cholesterol level. Insulin resistance has been found in persons with low levels of high-density lipoprotein. Insulin levels have also been linked to very-low-density lipoprotein synthesis and plasma triglyceride levels.

Accordingly, also disclosed are methods of treating insulin resistance in a subject comprising selecting a subject in need of treatment for insulin resistance; and administering to the subject an effective amount of a compound that inhibits MCT4.

Specific diseases to be treated by the compounds, compositions, and methods disclosed herein are those mediated at least in part by MCT4. Accordingly, disclosed herein are methods: for reducing glycogen accumulation in a subject; for raising HDL or HDLc, lowering LDL or LDLc, shifting LDL particle size from small dense to normal LDL, lowering VLDL, lowering triglycerides, or inhibiting cholesterol absorption in a subject; for reducing insulin resistance, enhancing glucose utilization or lowering blood pressure in a subject; for reducing visceral fat in a subject; for reducing serum transaminases in a subject; or for treating disease; all comprising the administration of a therapeutic amount of a compound as described herein, to a patient in need thereof. In further embodiments, the disease to be treated may be a metabolic disease. In further embodiment, the metabolic disease may be selected from the group consisting of obesity, diabetes melitus, especially Type 2 diabetes, hyperinsulinemia, glucose intolerance, metabolic syndrome X, dyslipidemia, hypertriglyceridemia, hypercholesterolemia, and hepatic steatosis. In other embodiments, the disease to be treated may be selected from the group consisting of cardiovascular diseases including vascular disease, atherosclerosis, coronary heart disease, cerebrovascular disease, heart failure and peripheral vessel disease. In preferred embodiments, the methods above do not result in the induction or maintenance of a hypoglycemic state.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Examples

The invention is further illustrated by the following Examples.

### Compound Synthesis

Compounds and salts can be prepared using methods illustrated in general synthetic schemes and experimental procedures detailed below. General synthetic schemes and experimental procedures are presented for purposes of illustration and are not intended to be limiting. Starting materials used to prepare compounds and salts described herein are commercially available or can be prepared using routine methods known in the art.

### Example 1:2-([1-[2-(azetidin-1-yl)phenyl]-5-(3-cyclobutoxyphenyl)-1H-pyrazol-3-yl]methoxy)-2-methylpropanoic acid

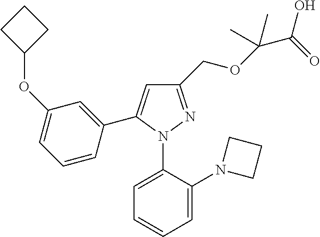 [SMILES: CC(C)(OCc1cc(-c2cccc(OC3CCC3)c2)n(-c2ccccc2N2CCC2)n1)C(=O)O]

A compound of structural Formula I (i.e., Compound 1) was synthesized by the process of Scheme 1.

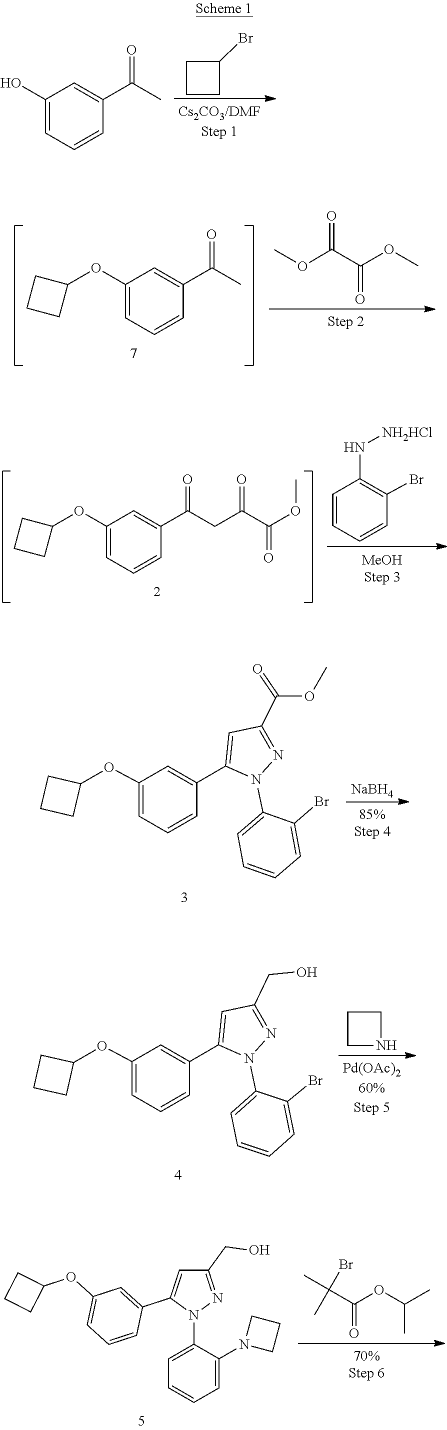

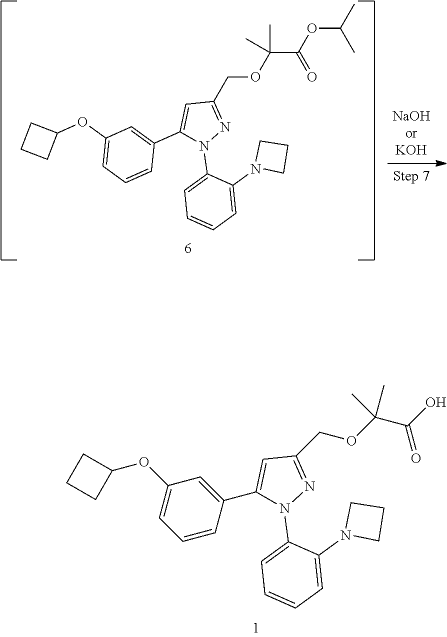

### Step 1:

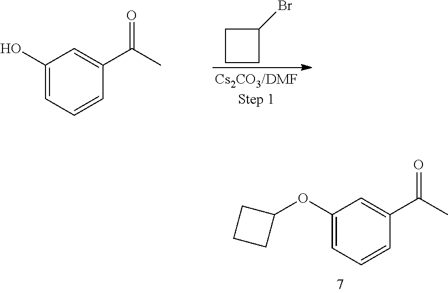

A mixture of 1-(3-hydroxyphenyl) ethan-1-one (20 kg), bromocyclobutane (1.3 eq.) and Cs₂CO₃ (1.5 eq.) in DMF (5 volumes) was stirred for 16 hours at 80±5° C. The reaction vessel was charged with water (15 volumes) and methyl tert-butyl ether (MTBE, 15 volumes). The organic layer was separated and washed twice with 20% brine (5 volumes), then concentrated until about 5 volumes and then solvent-switched with methanol three times until about 5 volumes. The crude methanolic solution containing compound 7 was used without further purification in the next step.

### Step 2:

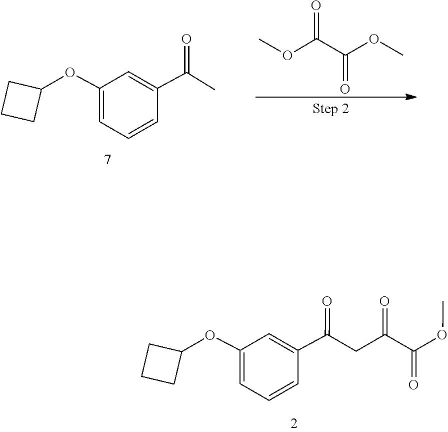

To the crude methanolic solution of Step 1 (5 volumes) was added sodium methoxide (2.0 eq.) and dimethyl oxalate (1.5 eq.). The reaction vessel was stirred for 16 hours at 30±5° C. Upon completion, the reaction mixture was cooled to a temperature between 0-10° C. The pH was adjusted to 2-3 with 4.0 M HCl in methanol, and the crude solution containing compound 2 was used without further purification in the next step.

### Step 3:

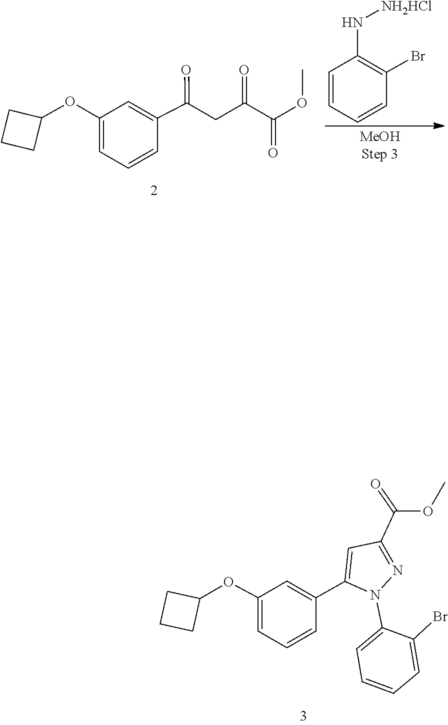

To the reaction vessel containing the crude methanolic solution of Step 2 was added 2-bromophenylhydrazine hydrochloride (1.0 eq.). The reaction mixture was then stirred for 10 hours at 60±5° C., then cooled to 5-15° C. The resulting solids were filtered, washed with methanol (1 volume), and then slurried with water (20 volumes). The solids were filtered again and further washed with water (2 volumes). The product was dried under 50° C. in an oven to afford 54.9 kg of Compound 3 (88% yield for steps 1-3).

### Step 4:

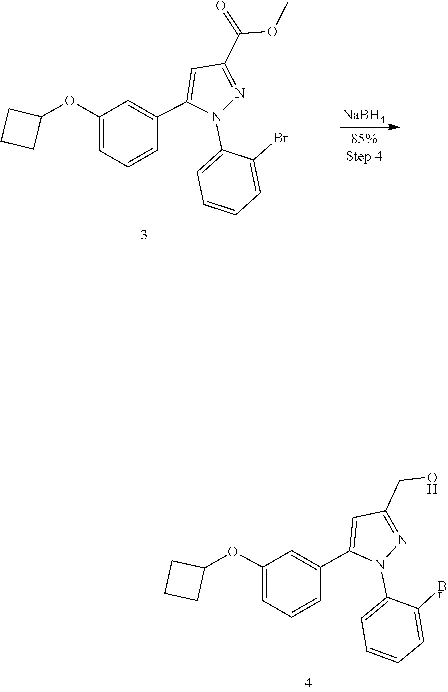 [SMILES: COC(=O)c1cc(-c2cccc(OC3CCC3)c2)n(-c2ccccc2Br)n1 ; "NaBH4" ; OCc1cc(-c2cccc(OC3CCC3)c2)n(-c2ccccc2Br)n1]

Compound 3 (4.0 kg; 1.0 eq.) was added to THF (4 volumes) before methanol (0.4 vol.) was further added to the reaction vessel which was then cooled to 10-25° C. Sodium borohydride (1.2 eq) was added, and the reaction was stirred for 16 hours at 10-25° C. Upon completion, the reaction was cooled to 0-10° C., and the pH was adjusted to 3-5 with 0.5 M HCl, at which point, the product precipitated out of solution. The mixture was stirred for an additional 1-2 hours at 5-15° C. The product was collected via filtration, washed with water, and dried under 50° C. in an oven to afford 3.5 kg of compound 4, 85% yield.

Step 4 was repeated at 5× scale with Compound 3 (20.0 kg; 1.0 eq.) added to THF (6.0 volumes) before methanol (0.4 vol.) was further added to the reaction vessel which was then cooled to 25±5° C. Sodium borohydride (1.2 eq) was added, and the reaction was stirred for 16 hours at 25±5° C. Upon completion, the reaction was cooled to 0-10° C., and the pH was adjusted to 3-5 with 0.5 M HCl, at which point, the product precipitated out of solution. The mixture was stirred for an additional 1-2 hours at 5-15° C. The product was collected via filtration, washed with water, and dried under 50° C. in an oven to afford 18.2 kg of Compound 4 at 99.4% purity and an isolated yield of 97%.

### Step 5:

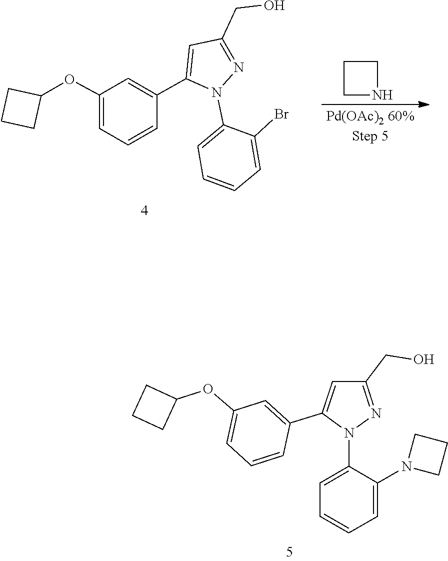

Compound 4 (2.97 kg; 1.0 eq.) was added with stirring and under a nitrogen atmosphere to THF (10 volumes). Xantphos (0.11 eq.) and Pd(OAc) 2 (0.11 eq.) and t-BuONa (2.0 eq.) were added to the reaction vessel. Azetidine (2.5 eq.) was added, and the resulting mixture was stirred for 20 hours at 25-35° C. The vessel was charged with MTBE (20 volumes) and water (20 volumes). The organic layer was separated, washed with 5% aqueous NH₄Cl and 5% aqueous NaCl and filtered through Celite®. The resulting organic layer was concentrated and then solvent-switched with ethyl acetate (10 volumes). The resulting solution was stirred for 303 minutes at 70-80° C., then cooled to 15-20° C. and stirred for an additional 1-2 hours. The solids were collected via filtration, washed with ethyl acetate (0.5 volumes), and dried under 50° C. in an oven to afford 1.7 kg of compound 5, 60% yield. When reproduced with 18.0 kg of Compound 4, 10.8 kg of Compound 5 were afforded, with a purity of 98.8% and an isolated yield of 63.8%.

### Step 6

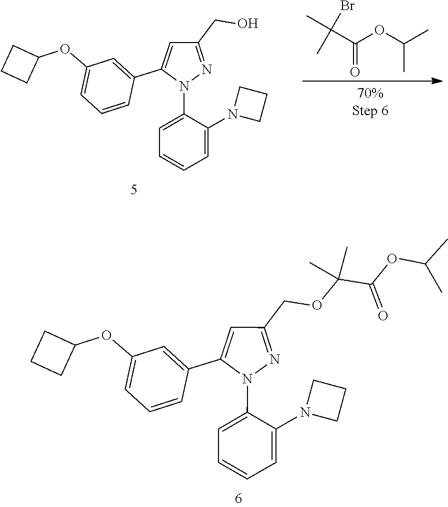

To a solution of compound 5 (700.0 g, 1 eq.) in DMF (8 volumes) was added isopropyl 2-bromo-2-methylpropanoate (8.0 eq.). The reaction mixture was cooled to −60° C.-−50° C. using liquid nitrogen. A THE solution of potassium bis(trimethylsilyl)amide (KHMDS, 5.0 eq.) was added dropwise over 1.5 hours; then the reaction was stirred for 30-50 minutes. Water (10 volumes) was added to the reaction vessel. The organic phase was collected, washed with NaCl (15%) (5 volumes) twice, and then concentrated. The crude compound 6 (70% yield) was used directly in the next step without further purification.

### Step 7

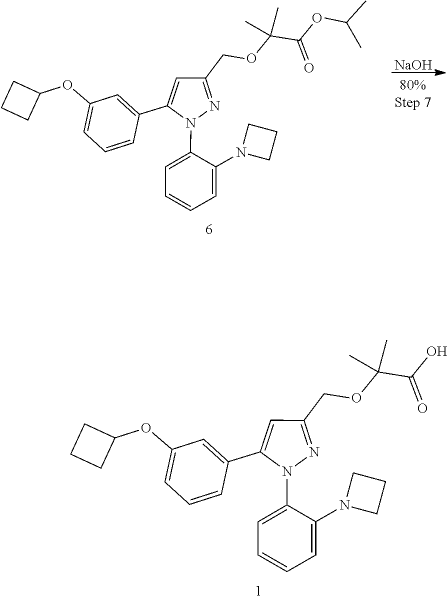

Crude Compound 6 (1950.0 g, 1 eq.) from step 6 was added to the reaction vessel with 30% (aq.) KOH (10 volumes, 20 eq.) and methanol (5 volumes). The reaction was stirred for 16 hours at 50±5° C., then cooled to 15-30° C. The solution was washed twice with MTBE (10 volumes), then the aqueous layer was separated and filtered through Celite®. The pH of the resulting aqueous phase was adjusted to 3-5 using 2.0 M HCl. The solution was cooled to 5-10° C. and stirred for 2-3 hours, then filtered. The filter cake was dissolved in THF (10 volumes), then mercapto silica gel (300 g, 15% w/w) was added to the solution. The resulting mixture was heated to 50-60° C., stirred for 3 hours, and then filtered. The solution was concentrated, then the crude product was dissolved in ethyl acetate (10 volumes). The resulting solution was heated to 70-80° C. and stirred for 3-4 hours, then cooled to 0-10° C. The solids were collected via filtration, washed with ethyl acetate (0.5 volumes), then dried under 50° C. in an oven to afford compound 1 (1.4 kg, 58% yield) as an off-white solid. ¹H NMR (C₂D₆OS) δ 12.66 (s, 1H), 7.20 (dd, 2H), 6.98 (t, 2H), 6.73 (m, 3H), 6.60 (d, 1H), 6.53 (d, 1H), 4.45 (s, 2H), 4.32 (t, 1H), 3.48 (dd, 4H), 3.32 (s, 3H), 2.27 (d, 2H), 2.05 (m, 2H), 1.93 (m, 2H), 1.73 (m, 1H), 1.56 (m, 1H), 1.415 (s, 6H).

### Example 2: Large Scale Synthesis of 2-([1-[2-(azetidin-1-yl)phenyl]-5-(3-cyclobutoxyphenyl)-1H-pyrazol-3-yl]methoxy)-2-methylpropanoic acid and tris salt

### 1. Synthesis of Compound 1

Compound 1 was synthesized by the process of Scheme 1.

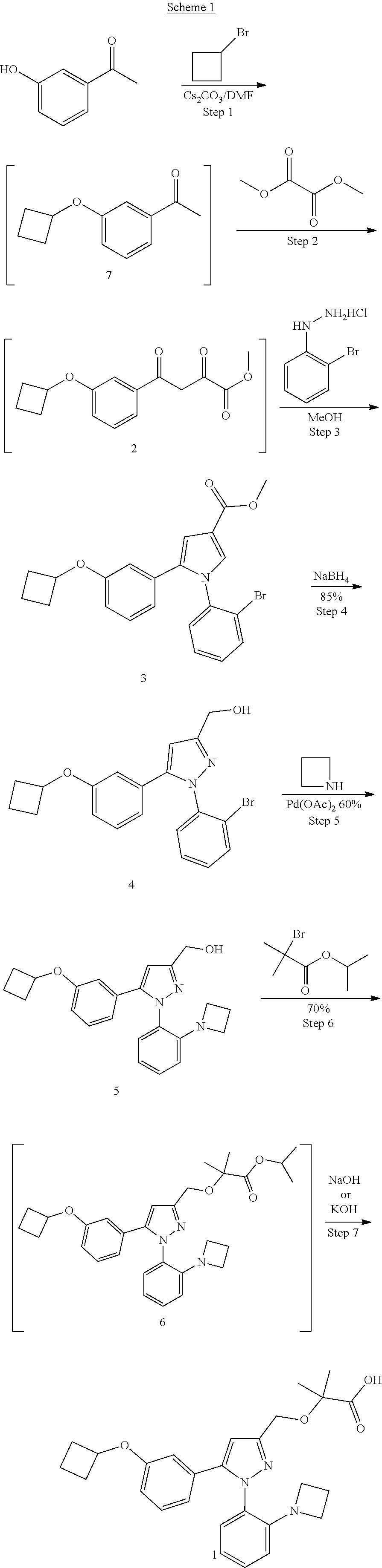

### Step 1:

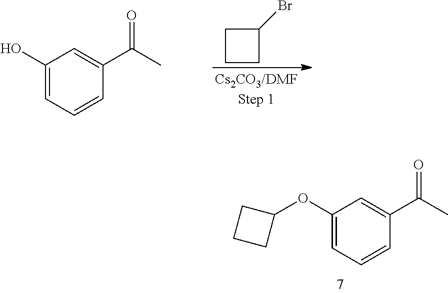

A mixture of 1-(3-hydroxyphenyl) ethan-1-one (20 kg), bromocyclobutane (1.3 eq.) and Cs₂CO₃ (1.5 eq.) in DMF (5 volumes) was stirred for 16 hours at 80±5° C. The reaction vessel was charged with water (15 volumes) and methyl tert-butyl ether (MTBE, 15 volumes). The organic layer was separated and washed twice with 20% brine (5 volumes), then concentrated until about 5 volumes and then solvent-switched with methanol three times until about 5 volumes. The crude methanolic solution containing Compound 7 was used without further purification in the next step.

### Step 2:

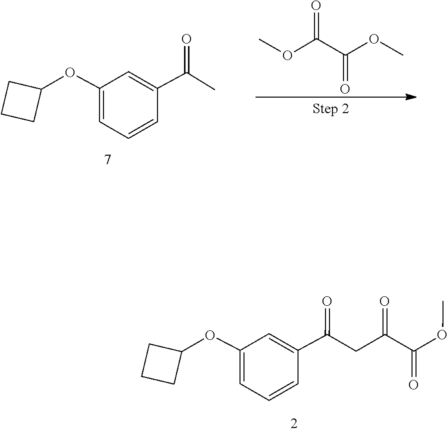

To the crude methanolic solution of Step 1 (5 volumes) was added sodium methoxide (2.0 eq.) and dimethyl oxalate (1.5 eq.). The reaction vessel was stirred for 16 hours at 30±5° C. Upon completion, the reaction mixture was cooled to a temperature between 0-10° C. The pH was adjusted to 2-3 with 4.0 M HCl in methanol, and the crude solution containing Compound 2 was used without further purification in the next step.

### Step 3:

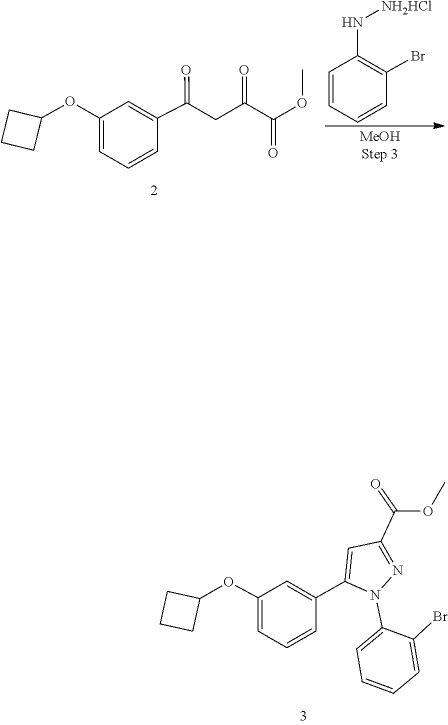

To the reaction vessel containing the crude methanolic solution of Step 2 was added 2-bromophenylhydrazine hydrochloride (1.0 eq.). The reaction mixture was then stirred for 10 hours at 60±5° C., then cooled to 5-15° C. The resulting solids were filtered, washed with methanol (1 volume), and then slurried with water (20 volumes). The solids were filtered again and further washed with water (2 volumes). The product was dried under 50° C. in an oven to afford 54.9 kg of Compound 3 (88% yield for steps 1-3).

### Step 4:

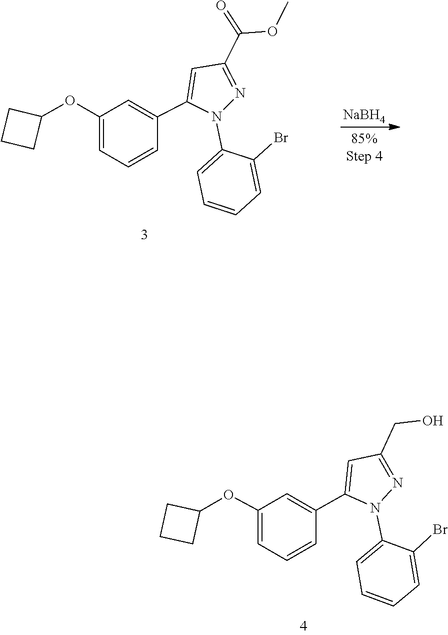 [SMILES: COC(=O)c1cc(-c2cccc(OC3CCC3)c2)n(-c2ccccc2Br)n1 ; "NaBH4" ; OCc1cc(-c2cccc(OC3CCC3)c2)n(-c2ccccc2Br)n1]

Compound 3 (4.0 kg; 1.0 eq.) was added to THF (4 volumes) before methanol (0.4 vol.) was further added to the reaction vessel which was then cooled to 10-25° C. Sodium borohydride (1.2 eq) was added, and the reaction was stirred for 16 hours at 10-25° C. Upon completion, the reaction was cooled to 0-10° C., and the pH was adjusted to 3-5 with 0.5 M HCl, at which point, the product precipitated out of solution. The mixture was stirred for an additional 1-2 hours at 5-15° C. The product was collected via filtration, washed with water, and dried under 50° C. in an oven to afford 3.5 kg of Compound 4, 85% yield.

Step 4 was repeated at 5× scale with Compound 3 (20.0 kg; 1.0 eq.) added to THF (6.0 volumes) before methanol (0.4 vol.) was further added to the reaction vessel which was then cooled to 25±5° C. Sodium borohydride (1.2 eq) was added, and the reaction was stirred for 16 hours at 25±5° C. Upon completion, the reaction was cooled to 0-10° C., and the pH was adjusted to 3-5 with 0.5 M HCl, at which point, the product precipitated out of solution. The mixture was stirred for an additional 1-2 hours at 5-15° C. The product was collected via filtration, washed with water, and dried under 50° C. in an oven to afford 18.2 kg of Compound 4 at 99.4% purity and an isolated yield of 97%.

### Step 5:

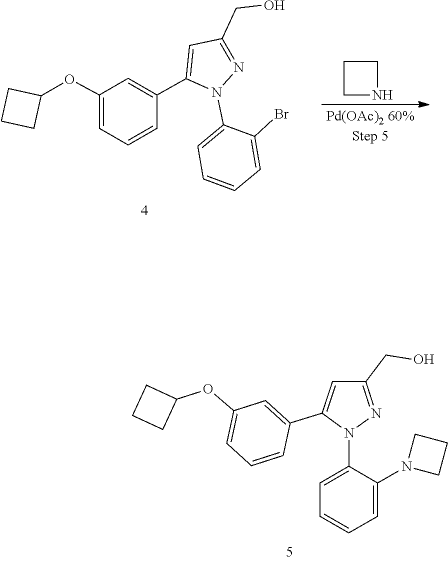

Compound 4 (2.97 kg; 1.0 eq.) was added with stirring and under a nitrogen atmosphere to THF (10 volumes). Xantphos (0.11 eq.) and Pd(OAc) 2 (0.11 eq.) and t-BuONa (2.0 eq.) were added to the reaction vessel. Azetidine (2.5 eq.) was added, and the resulting mixture was stirred for 20 hours at 25-35° C. The vessel was charged with MTBE (20 volumes) and water (20 volumes). The organic layer was separated, washed with 5% aqueous NH₄Cl and 5% aqueous NaCl and filtered through Celite®. The resulting organic layer was concentrated and then solvent-switched with ethyl acetate (10 volumes). The resulting solution was stirred for 303 minutes at 70-80° C., then cooled to 15-20° C. and stirred for an additional 1-2 hours. The solids were collected via filtration, washed with ethyl acetate (0.5 volumes), and dried under 50° C. in an oven to afford 1.7 kg of Compound 5, 60% yield. When reproduced with 18.0 kg of Compound 4, 10.8 kg of Compound 5 were afforded, with a purity of 98.8% and an isolated yield of 63.8%.

### Step 6

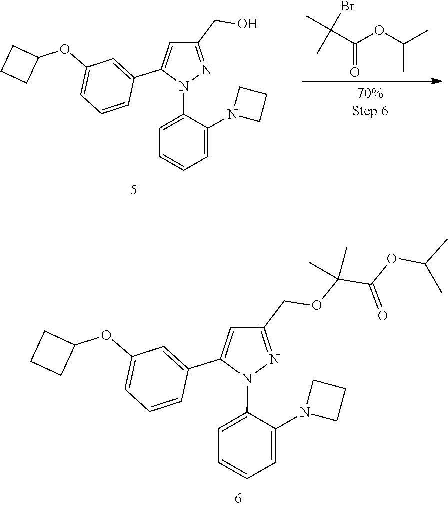

To a solution of Compound 5 (700.0 g, 1 eq.) in DMF (8 volumes) was added isopropyl 2-bromo-2-methylpropanoate (8.0 eq.). The reaction mixture was cooled to between −60° C. and −50° C. using liquid nitrogen. A THF solution of potassium bis(trimethylsilyl)amide (KHMDS, 5.0 eq.) was added dropwise over 1.5 hours; then the reaction was stirred for 30-50 minutes. Water (10 volumes) was added to the reaction vessel. The organic phase was collected, washed with NaCl (15%) (5 volumes) twice, and then concentrated. The crude Compound 6 (70% yield) was used directly in the next step without further purification.

### Step 7

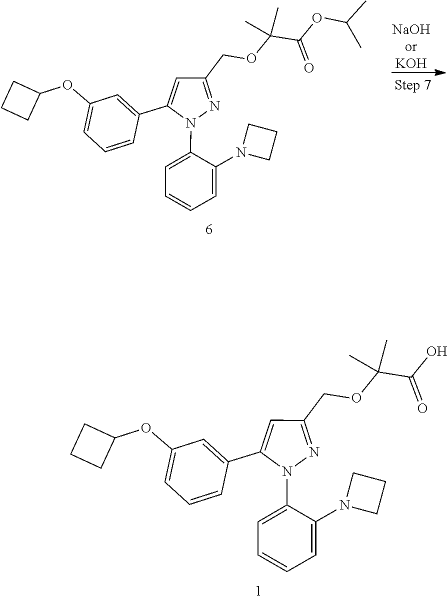 [SMILES: CC(C)OC(=O)C(C)(C)OCc1cc(-c2cccc(OC3CCC3)c2)n(-c2ccccc2N2CCC2)n1 ; "NaOHorKOH" ; CC(C)(OCc1cc(-c2cccc(OC3CCC3)c2)n(-c2ccccc2N2CCC2)n1)C(=O)O]

Crude Compound 6 (1950.0 g, 1 eq.) from step 6 was added to the reaction vessel with 30% (aq.) NaOH (10 volumes, 20 eq.) and methanol (5 volumes). The reaction was stirred for 16 hours at 50±5° C., then cooled to 15-30° C. The solution was washed twice with MTBE (10 volumes), then the aqueous layer was separated and filtered through Celite®. The pH of the resulting aqueous phase was adjusted to 3-5 using 2.0 M HCl. The solution was cooled to 5-10° C. and stirred for 2-3 hours, then filtered. The filter cake was dissolved in THF (10 volumes), then mercapto silica gel (300 g, 15% w/w) was added to the solution. The resulting mixture was heated to 50-60° C., stirred for 3 hours, and then filtered. The solution was concentrated, then the crude product was dissolved in ethyl acetate (10 volumes). The resulting solution was heated to 70-80° C. and stirred for 3-4 hours, then cooled to 0-10° C. The solids were collected via filtration, washed with ethyl acetate (0.5 volumes), then dried under 50° C. in an oven to afford Compound 1 (1.4 kg, 58% yield) as an off-white solid.

This reaction was repeated at a larger scale with crude Compound 6 (8.4 kg, 1 eq.) from step 6 was added to the reaction vessel with 30% (aq.) KOH (10 volumes, 20 eq.) and methanol (5 volumes). After following the same workup as above, 8.76 kg of Compound 1 were afforded at a purity of 97.7% and a yield of 67% over two steps.

### 2. Use of Different Propanoate Esters in Step 6

Varying the propanoate ester in step 6 was explored. The results are shown in Table 1.

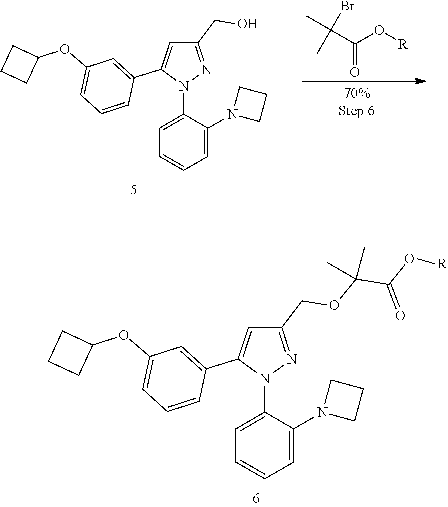

TABLE 1

| | | | Reaction | | |
|---|---|---|---|---|---|
| | Starting Materials | | Conditions | | IPC |
| Trial | and Reagents | | Temp. | Time | Starting |
| # | R | Reagents | ° C. | (h) | Material:Product |
| 1 | Methyl | NaH (4.0 eq.) | 0~15 | 16 | 6.0%:82.5% |
| | | DMF (10.0 v) | | | |
| 2 | Methyl | NaH (4.5 eq.) | 0~10 | 18 | 6.7%:46.2% |
| | | DME (10.0 v) | | | |
| 3 | Methyl | NaH (4.5 eq.) | 0~10 | 18 | 9.9%:62.9% |
| | | NMP (10.0 V) | | | |
| 4 | Methyl | tBuONa (4.0 eq.) | 0~15 | 16 | 15.9%:39.5% |
| | | DMF (10.0 V) | | | |
| 5 | Methyl | Sodium tert- | 0~15 | 16 | 19.9%:40.7% |
| | | pentoxide (4.0 eq.) | | | |
| | | DMF (10.0 V) | | | |
| 6 | Methyl | Cs₂CO₃ (6.0 eq.) | 20~30 | 18 | 83.5%:11.6% |
| | | DMF (10.0 V) | | | |
| 7 | Methyl | NaI(0.1 eq.) | 50~55 | 18 | 47.0%:37.7% |
| | | Cs₂CO₃(6.0 eq.) | | | |
| | | DMF (10.0 V) | | | |
| 8 | Methyl | Cs₂CO₃ (6.0 eq.) | 80~90 | 36 | 34.4%:33.7% |
| | | DMF (10.0 V) | | | |
| 9 | t-Butyl | NaH (4.0 eq.) | 0~15 | 18 | 9.9%:34.7% |
| | | DMF (10.0 V) | | | |
| 10 | t-Butyl | tBuONa (4.0 eq.) | 0~15 | 18 | 33.0%:26.0% |
| | | DMF (10.0 V) | | | |
| 11 | t-Butyl | Sodium tert- | 0~15 | 18 | 36.4%:25.5% |
| | | pentoxide (4.0 eq.) | | | |
| | | DMF (10.0 V) | | | |
| 12 | Methyl | LiHMDS (4.5 eq.) | 0~10 | ~18 | 98.2%:0% |
| | | THF (10.0 v) | | | |
| 13 | Methyl | KHMDS (1.0 eq.) | −50~−40 | 1.5~2 | 31.5%:45.0%* |
| | | DMF (10.0 v) | | | |
| 14 | Isopropyl | KHMDS (6.0 eq.) | −60~−50 | 1.5~2 | 4.9%:84.5% |
| | | DMF (20.0 V) | | | |
| 15 | Isopropyl | DMF (20.0 V) | −60~−50 | Add 5 | 52.6%:38.3% |
| | | | | mins+ | |
| | | KHMDS (1.2 eq.) | | Stir | |
| | | | | 15~20 | |
| | | | | mins | |
| 16 | Isopropyl | KHMDS (1.2 eq.), | −60~−50 | Add 5 | 16.8%:72.3% |
| | | Total 2.4 eq. | | mins+ | |
| | | | | Stir | |
| | | | | 15~20 | |
| | | | | mins | |
| 17 | Isopropyl | KHMDS (1.2 eq.), | −60~−50 | Add 5 | 6.2%:82.5% |
| | | Total 3.6 eq. | | mins+ | |
| | | | | Stir | |
| | | | | 15~20 | |
| | | | | mins | |
| 18 | Isopropyl | KHMDS (1.2 eq.), | −60~−50 | Add 5 | 4.5%:88.3% |
| | | Total 4.8 eq. | | mins+ | |
| | | | | Stir | |
| | | | | 15~20 | |
| | | | | mins | |
| 19 | Isopropyl | KHMDS (1.2 eq.), | −60~−50 | Add 5 | 3.2%:89.6% |
| | | Total 6.0 eq. | | mins+ | |
| | | | | Stir | |
| | | | | 15~20 | |
| | | | | mins | |

*Additionally, 13.4% of a dimer side product was detected upon completion of the reaction.

As shown in Table 1, the starting material with the isopropyl ester performed better than the t-butyl ester and methyl ester. Additionally, use of the isopropyl ester limits the potential formation of a dimer (or bis-adduct) side product, which was detected when using the methyl ester in Trial 13. Use of the isopropyl ester also permits the use of a solvent other than DMF/NaH. The reaction also proceeded much more quickly than with DMF/NaH.

### 3. Formation of Tris Salt of Compound 1

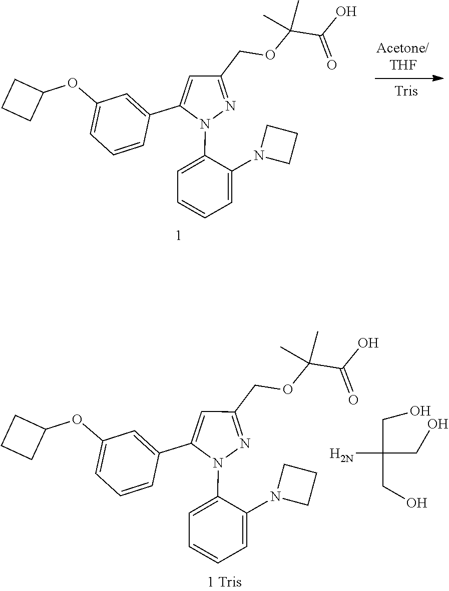

Compound 1 (8.4 kg, 1 eq.) was added to a reaction vessel with THF (42 L, 5.0 volumes), charged with mercapto silica gel (420 g, 5 wt. %), and stirred and heated at 50-60° C. for three hours. The reaction mixture was sampled and concentrated for ICP analysis to determine the residual Pd concentration (˜15 ppm). The reaction mixture was filtered charged with acetone (168 L, 20.0 volumes) and tris(hydroxymethyl)aminomethane (“Tris”, 1.01 eq.) solution in water (4.2 L, 0.5 volumes). The reaction mixture was stirred for 5-10 minutes at 20-30° C. for most of the solids to dissolve and continued to stir for about 20 hours at 20-30° C. to permit the Tris salt of Compound 1 to precipitate out. The precipitant was filed, washed with acetone (4.2 L, 0.5 volumes) and dried under vacuum at 60° C. to afford 8.4 kg of Compound 1 Tris at 99.5% purity and a yield of 79.2%, having 1690 ppm residual acetone and 962 ppm residual THF.

### Example 3: Salt Formulation

### Methods

Polarized light microscopy (PLM) was performed with Olympus BX60 polarized-light microscope equipped with Olympus DP70 camera.

X-ray powder diffraction (“XRPD”) was performed with a PANalytical X'Pert Pro diffractometer using Ni-filtered Cu Ka (45 kV/40 mA) radiation and a step size of 0.02° 2Θ and X'celerator™ RTMS (Real Time Multi-Strip) detector. Configuration on the incidental beam side: fixed divergence slit) (0.25°), 0.04 rad Soller slits, anti-scatter slit) (0.25°), and 10 mm beam mask. Configuration on the diffracted beam side: fixed divergence slit) (0.25°) and 0.04 rad Soller slit. Samples were mounted flat on zero-background Si wafers.

Differential scanning calorimetry (“DSC”) was performed with a TA Instruments Q100 differential scanning calorimeter equipped with an autosampler and a refrigerated cooling system under 40 mL/min N₂ purge. DSC thermograms were obtained at 15° C./min in crimped A1 pans.

Thermogravimetric analysis (“TGA”) was performed with a TA Instruments Q500 thermogravimetric analyzer under 40 mL/min N₂ purge at 15° C./min in Pt or A1 pans.

Thermogravimetric Analysis with IR Off-Gas Detection (TGA-IR) was conducted with a TA Instruments Q5000 thermogravimetric analyzer interfaced to a Nicolet 6700 FT-IR spectrometer (Thermo Electron) equipped with an external TGA-IR module with a gas flow cell and DTGS detector. TGA was conducted with 60 mL/min N₂ flow and heating rate of 15° C./min in Pt or A1 pans. IR spectra were collected at 4 cm⁻¹ resolution and 32 scans at each time point.

The ¹H NMR spectra were collected using Agilent DD2 500 MHz spectrometer with TMS reference. Samples were dissolved in DMSO-d6.

Ion Chromatography (IC) was performed on a Dionex ICS-5000. Column: Dionex IonPac CS12 4×250 mm; Detection: Suppressed conductivity, CERS 500 with suppressor current at 59 mA; Eluent (20 mM Methanesulfonic acid) at 1.0 mL/min.

Preliminary Salt Formation Experiments with the Example 1 Compound

The following general procedure was used to conduct salt experiments with Example 1. About 20 mg of the Example 1 compound was weighed into individual vials. Either 200 μL or 1000 μL of solvent was added to an individual vial along with a stoichiometric amount of the counterion. The resulting solutions/suspensions/gums were stirred while cycling the temperature between 40° C. and 5° C. for two days (TC1). The solvents of gums/solutions after TC1 were evaporated under reduced pressure, and then 200 μL of the solvent was re-dispensed into the vial. The resulting solutions/suspensions/gums were again stirred while cycling the temperature between 40° C. and 5° C. for two days (TC2). The solutions/gums/gels were cooled rapidly to 4° C. and held at 4° C. for two days (RC). The solvent was then evaporated at ambient conditions for seven days (SEV). Samples were examined for birefringence by PLM in situ at each step, and if birefringent, isolated, analyzed and grouped by FT-Raman and/or XRPD. A representative sample for each group was also characterized by DSC. The samples with promising DSC results were further characterized by TGA-IR, XRPD, HNMR and/or IC.

TABLE 2
Summary of salt experiments.

| | Solvent | | | | | |
|---|---|---|---|---|---|---|
| | | | | | | THF/20% |
| Counterion | Acetone | i-PrOH | MeCN | EtOAc | MTBE | Water |
| KOH (1 eq) | A | A | A | P | A | A |
| L-Arginine 1(eq) | A | A | A | A | A | A |
| Ca(OAc)₂ (0.5 eq) | B | A | P/C | P | P | A |
| L-Lysine (1 eq) | A | A | A | A | A | A |
| N-Methyl-D- | A | A | A | A | A | A |
| Glucamine (1 eq) | | | | | | |
| Tris (1 eq) | P | A | B | A | A | A |
| HCl (1 eq) | A | A | A | A | A | A |
| Naphthalene-1,5- | A | A | A | A | A | A |
| disulfuric acid (0.5 eq) | | | | | | |
| Sulfuric Acid (1 eq) | A | A | A | A | A | A |
| Ethanesulfonic Acid | A | A | A | A | A | A |
| (1 eq) | | | | | | |
| p-Toluenesulfonic | A | A | A | A | A | A |
| Acid (1 eq) | | | | | | |
| Methanesulfonic Acid | A | A | A | A | A | A |
| (1 eq) | | | | | | |
| Benzenesulfonic Acid | A | A | A | A | A | A |
| (1 eq) | | | | | | |
| L-Aspartic Acid (1 eq) | P/C | P/C | P/C | P/C | P/C | C |
| Phosphoric Acid (1 eq) | P | P | A | P | P | P |
| 1-Hydroxy-2- | P | P | P | P | P | A |
| naphthoic acid (1 eq) | | | | | | |

A = Amorphous/Gum
B = Birefringent Hit
P = Crystalline Parent
C = Counterion

Compound 1 tris salt Form A (4088.62 mg) was transferred to an 125-mL Erlenmeyer flask and 70 mL of solvent (95:5 acetone: water v/v) added and stirred with a magnetic stir bar. After stirring for 5 minutes, Compound 1 tris salt Form B seed produced from the primary polymorph screen described above (53.19 mg) was added and stirring continued. After stirring for 15 min, the sample solids had seized, and the stirrer bar was no longer moving. The flask was hand-shook to break up mass. A small aliquot was obtained and analyzed by PXRD which indicated the solids were Form B. The remaining solids were isolated on Büchner funnel with a Whatman #1 filter paper. The solids were washed with 20 mL of acetone chilled at −20° C. The funnel with isolated solids was covered with Kimwipe and left to dry on filter with vacuum applied overnight. NMR indicated the Compound 1: tris molar ratio was 1:1. The chromatographic purity was 99.8% area (254 nm).

After approximately 14 hours of drying, the solids were isolated (3804.50 mg, 93% yield). PXRD confirmed sample was Form B tris salt with no detectable Form A tris salt and no detectable Compound 1 parent. The sample exhibited negligible weight loss prior to thermal decomposition by TGA.

### Characterization of Form A Tris Salt

One crystalline tris salt was isolated from six experiments during the salt studies. The hit was designated as Form A tris salt. The remaining experiments yielded either parent Form A or amorphous/gums. Thermal analysis indicated that Form A tris was a non-solvated form.

DSC data showed a melting endotherm with onset at 147.4° C. TGA-IR analysis showed negligible weight loss (0.3%) of water between 25-150° C., indicating the salt is non-solvated. ¹H-NMR indicated the stoichiometry is a mono-salt (ratio of API/CI is 1:1).

### Characterization of the Form B Tris Salt

The Form B tris salt is an off-white crystalline powder consisting of small particles. By PXRD, it shows relatively sharp diffraction peaks between 2-40° 2Θ consistent with a crystalline material. DSC analysis showed a melt/decomposition endotherm with onset at 148° C. (ΔH=111 J/g). TGA analysis showed negligible (0.1%) total weight loss up to 145° C. indicating that Form B is non-solvated. DVS showed the API to be non-hygroscopic with weight change of approximately 0.1% at 25° C., between 5 and 95% RH. Sample recovered after the dynamic vapor sorption experiment did not indicate a change in the solid form by PXRD.

Kinetic solubility was assessed in biorelevant media at room temperature at 1, 4 and 24 hours, and summarized in Table 3. Form B tris salt was practically insoluble in fasted-state simulated gastric fluid (FaSSGF) after 1, 4 and 24 hours (0.46, 0.35 μg/mL and 0.39 μg/mL, respectively). Solubility in fasted-state simulated intestinal fluid (FaSSIF) was at least at 3 mg/mL (parent equivalent), but gradually decreased from 1 hour (1112 μg/mL), 4 hours (851 μg/mL) to 24 hours (733 μg/mL).

Fed-state simulated intestinal fluid (FeSSIF) kinetic solubility was also in solution at 3 mg/mL (parent equivalent), then precipitated (99 μg/mL at 1 hour, 96 μg/mL at 4 hours and 95 μg/mL at 24 hours).

TABLE 3
Kinetic Solubility of tris salt Form B in Biorelevant Media

| | | Solubility (μg/mL | | | pH of Sample | | | |
|---|---|---|---|---|---|---|---|---|
| | | parent equiv.) | | | Filtrate | | | |
| | Media | t = 1 | t = 4 | t = 24 | t = 1 | t = 4 | t = 24 | |
| Media | pH | hour | hours | hours | hour | hours | hours | XRPD |
| FaSSGF | 1.5 | 0.46 | 0.35 | 0.39 | 1.55 | 1.60 | 1.64 | Parent |
| pH 1.6 | | | | | | | | Form A |
| FaSSIF | 6.55 | 1112 | 851 | 733 | 7.61 | 7.67 | 7.73 | Parent |
| #2 pH 6.5 | | | | | | | | Form A |
| FeSSIF | 5.84 | 99 | 96 | 95 | 6.03 | 6.13 | 6.03 | Parent |
| #2 pH 5.8 | | | | | | | | Form A |

Stability of the solid API was assessed after storage at the following conditions for 2 weeks:
25° C./58% RH (closed)
25° C./58% RH (open)
40° C./75% RH (closed)
40° C./75% RH (open)
80° C./ambient RH (closed)

No significant chemical or physical changes were observed for tris salt Form B samples stored at 25° C./58% RH (closed and open), 40° C./75% RH (closed and open), and 80° C./ambient RH (closed) for 2 weeks. PXRD analysis of all stability samples also showed no significant change. Results of assessment of the solid-state stability of API are summarized Table 4.

TABLE 4
Summary of Solid-State Stability Assessment for tris salt Form B -2 Weeks

| | | | | Impurity (% area) | | | | | | |
|---|---|---|---|---|---|---|---|---|---|---|
| | | Main Peak | Content | RRT | RRT | RRT | RRT | RRT | Total | |
| Conditions | Time | (% Area) | (% w/w) | 0.60 | 0.86 | 0.89 | 0.92 | 1.21 | Impurities | XRPD |
| Initial | Initial | 99.81 | 101.2 | <0.05 | <0.05 | <0.05 | 0.13 | <0.05 | 0.19 | Consistent with Tris salt (Grp B) |
| 25° C. - | 2 | 99.78 | 101.1 | <0.05 | <0.05 | <0.05 | 0.13 | <0.05 | 0.22 | Consistent with Tris salt (Grp B) |
| 58% RH | weeks | | | | | | | | | input API |
| (c) | | | | | | | | | | |
| 25° C. - | 2 | 99.78 | 101.0 | <0.05 | <0.05 | <0.05 | 0.13 | <0.05 | 0.22 | Consistent with Tris salt (Grp B) |
| 58% RH | weeks | | | | | | | | | input API |
| (o) | | | | | | | | | | |
| 40° C. - | 2 | 99.75 | 101.3 | <0.05 | <0.05 | <0.05 | 0.13 | <0.05 | 0.25 | Consistent with Tris salt (Grp B) |
| 75% RH | weeks | | | | | | | | | input API |
| (c) | | | | | | | | | | |
| 40° C. - | 2 | 99.77 | 101.1 | <0.05 | <0.05 | <0.05 | 0.13 | <0.05 | 0.23 | Consistent with Tris salt (Grp B) |
| 75% RH | weeks | | | | | | | | | input API |
| (o) | | | | | | | | | | |
| 80° C. | 2 | 99.75 | 101.0 | <0.05 | <0.05 | <0.05 | 0.13 | <0.05 | 0.25 | Consistent with Tris salt (Grp B) |
| (c) | weeks | | | | | | | | | input API |

### Pharmacokinetic Assays

The tris salt of Compound 1 and the free acid were tested in two animal models to assess pharmacokinetic parameters.

### Rat Model

Groups, Dosing, and Collection. The pharmacokinetics of the free acid and the tris salt of Compound 1 by oral gavage were assessed in male Sprague-Dawley rats. Three rats per group were perorally administered 20, 60, or 200 mg Compound 1 free acid or 25, 75, or 250 mg in 0.5% methylcellulose in Saline for a final concentration of 2, 6, or 20 mg/mL Compound 1 free acid or 2.5, 7.5, or 20 mg/mL. Plasma was collected at 5 min, 15 min, 30 min, 1 hour, 2 hour, 4 hour, 6 hour, 8 hour, and 24 hour post dose via jugular vein. No abnormal clinical symptoms were observed.

Stock and Dose Prep. Stock solutions were prepared by dissolving 2.47 mg of Compound 1 (free acid) in 2.470 mL DMSO with vortexing to obtain 1 mg/mL solution of Compound 1 (free acid), or 2.09 mg of Compound 1 (Tris salt) in 1.655 mL DMSO with vortexing to obtain 1 mg/mL solution of Compound 1 (Tris salt). Dosing solutions were prepared by vortexing/sonicating the following solids in solvent:

TABLE 5
Preparation of Dosing Solutions

| Dose, mg/mL | Solid | Solvent |
|---|---|---|
| 2 | 40.1 mg free acid | 20.050 mL 0.5% MC in saline |
| 6 | 119.26 free acid | 19.877 mL 0.5% MC in saline |
| 20 | 396.69 free acid | 19.835 mL 0.5% MC in saline |
| 2.5 | 62.94 tris salt | 19.942 mL 0.5% MC in saline |
| 7.5 | 180.32 tris salt | 19.044 mL 0.5% MC in saline |
| 25 | 595.39 tris salt | 18.864 mL 0.5% MC in saline |

LC MS-MS Analysis. Liquid Chromatography with tandem mass spectrometry was used to determine plasma concentrations of Compound 1 Free Acid and Compound 1 Tris Salt in plasma samples collected at prescribed time points.

Appropriate serial concentrations of working solutions were achieved by diluting stock solution of analyte with 50% acetonitrile in water solution. Five μL of working solutions (10, 20, 50, 100, 500, 1000, 5000, 8000, 10000 ng/mL) were added to 50 μL of the blank male SD Rat plasma to achieve calibration standards of 1˜1000 ng/ml (1, 2, 5, 10, 50, 100, 500, 800, 1000 ng/mL) in a total volume of 55 μL. Five quality control (QC) samples at 2 ng/mL, 5 ng/mL, 10 ng/mL, 50 ng/mL and 800 ng/mL for plasma were prepared independently of those used for the calibration curves. These QC samples were prepared on the day of analysis in the same way as calibration standards. 55 μL standards, 55 μL QC samples and 55 μL unknown samples (50 μL plasma/blood with 5 μL blank solution) were added to 200 μL of methanol containing internal standard (dexamethasone) mixture for precipitating protein respectively, and vortexed for 30 seconds. After centrifugation at 4° C. and 4000 rpm for 15 minutes. The supernatant was diluted with water with a ratio of 1:2. 2 μL of the supernatant was injected into the LC/MS/MS system for quantitative analysis.

Instrumentation included HALO 90A C18 2.7 μm 2.1×50 mm HPLC column, a Prominence degasser DGU-20A5R(C), liquid chromatograph Shimadzu LC-30AD with communications bus module CBM-20A and Auto SIL-20AC HT; and an AB Sciex Triple Quad 5500 LC/MS/MS instrument. The following conditions were used:

### Mobile Phase

Solution A: 5% Acetonitrile in Water (0.1% Formic acid)
Solution B: 95% Acetonitrile in Water (0.1% Formic acid)

### Gradient

Flow rate: 0.5 mL/min

| Time (min) | A (%) | B (%) |
|---|---|---|
| 0.20 | 95.0 | 5.00 |
| 1.50 | 0.00 | 100 |
| 2.00 | 0.00 | 100 |
| 2.01 | 95.0 | 5.00 |
| 2.50 | 95.0 | 5.00 |

Injection volume: 2 μL

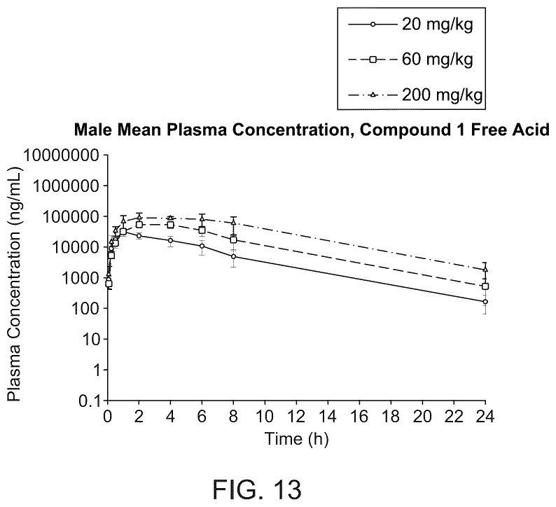

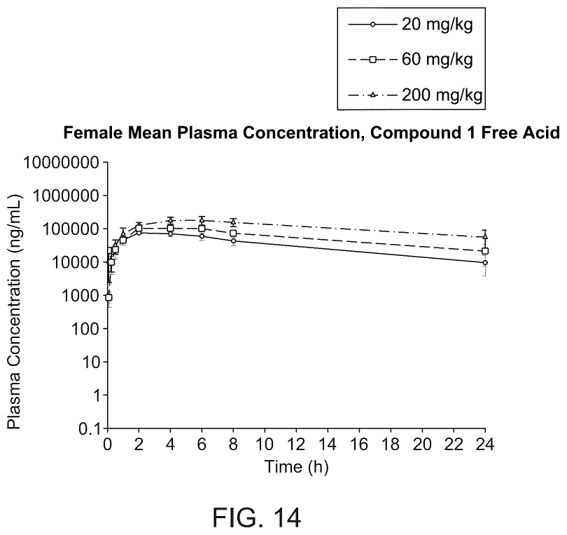

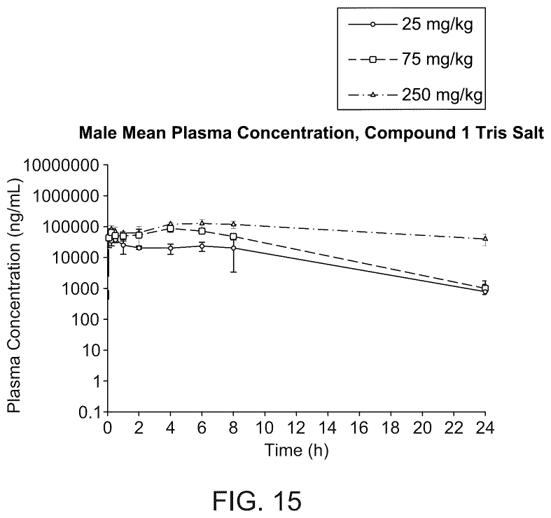

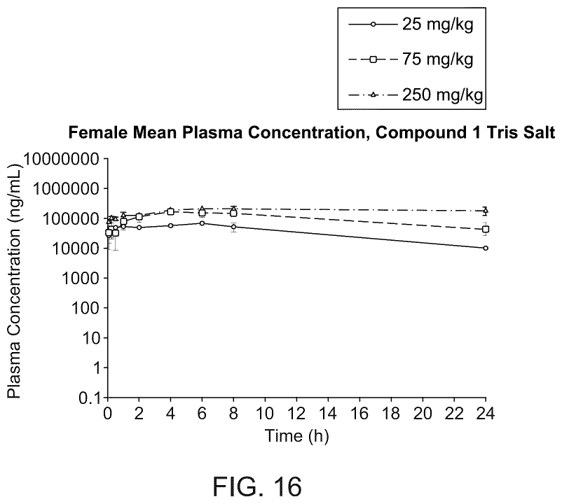

Results. Results are given below in Table 6 (free acid) and Table 7 (tris salt) and FIGS. 13-16. Note that because the formula weight is higher for the tris salt (FW=582.7) than for the free acid (MW=461.6), the appropriate comparison is 20 mg/kg free acid to 25 mg/kg tris salt. Overall, the total drug exposure across time (AUCₗₐₛₜ and AUC_{Inf}) was higher in both male and female subjects in the tris salt groups than in the free acid groups. Maximum exposure levels (Cₘₐₓ) were also higher in both male and female subjects in the tris salt groups than in the free acid groups, with the exception of females dosed at 20/25 mg/kg, where the free acid Cₘₐₓ (78,883 ng/mL) was slightly higher than the tris salt Cₘₐₓ (68,567 ng/mL). In general, exposure was higher in female subjects than male subjects.

TABLE 6
PK Parameters, Free Acid

| | 20 | 60 | 200 | 20 | 60 | 200 |
|---|---|---|---|---|---|---|
| | mg/kg, | mg/kg, | mg/kg, | mg/kg, | mg/kg, | mg/kg, |
| | male: | male: | male: | female: | female: | female: |
| | mean | mean | mean | mean | mean | mean |
| | (SD, | (SD, | (SD, | (SD, | (SD, | (SD, |
| PK parameter | CV %) | CV %) | CV %) | CV %) | CV %) | CV %) |
| T_{1/2}, h | 3.27 | 2.92 | 3.14 | 7 | 9.1 | 10.4 |
| | (1.05, | (0.42, | (0.34, | (1.71, | (5, | (3.6, |
| | 32.3) | 14.6) | 10.8) | 24.5) | 54.6) | 34.8) |
| Tₘₐₓ, h | 1 | 2.67 | 4 | 2.67 | 4.67 | 4.67 |
| | (0, | (1.15, | (2, | (1.15, | (2.31, | (1.15, |
| | 0) | 43.3) | 50) | 43.3) | 49.5) | 24.7) |
| Cₘₐₓ, ng/mL | 30633 | 56500 | 114533 | 78833 | 113000 | 206000 |
| | (2954, | (8805, | (19433, | (8456, | (15716, | (25981, |
| | 9.64) | 15.6) | 17 | 10.7) | 13.9) | 12.6) |
| AUCₗₐₛₜ, | 166649 | 445134 | 1104950 | 884621 | 1425555 | 2852952 |
| h*ng/mL | (35652, | (129431, | (348568, | (206245, | (127537, | (613410, |
| | 21.4) | 29.1) | 31.5) | 23.3) | 8.95) | 21.5) |
| AUC_{Inf}, | 167534 | 447474 | 1113663 | 990680 | 1770576 | 3815512 |
| h*ng/mL | (34931, | (131210, | (354633, | (280475, | (506147, | (1390909, |
| | 20.9) | 29.3) | 31.8) | 28.3) | 28.6) | 36.5) |
| AUC_{_{—}}_{% Extrap}_{_{—}}obs, | 0.61 | 0.477 | 0.704 | 9.9 | 16.7 | 22.1 |
| % | (0.68, | (0.313, | (0.406, | (5.6, | (14.6, | (13.2, |
| | 110) | 65.7) | 57.6) | 56.8) | 87.2) | 59.8) |
| MRT_{Inf}_{_{—}}obs, h | 4.54 | 5.33 | 6.01 | 10 | 13.6 | 15.9 |
| | (0.49, | (0.66, | (1.34, | (2.4, | (6.7, | (6.1, |
| | 10.9) | 12.4) | 22.4) | 23.9) | 49.3) | 38.4) |
| AUCₗₐₛₜ/D, | 8332 | 7419 | 5525 | 44231 | 23759 | 14265 |
| h*ng/mL | (1783, | (2157, | (1743, | (10312, | (2126, | (3067, |
| | 21.4) | 29.1) | 31.5) | 23.3) | 8.95) | 21.5) |

TABLE 7
PK Parameters, Tris Salt

| | 25 | 75 | 250 | 25 | 75 | 250 |
|---|---|---|---|---|---|---|
| | mg/kg, | mg/kg, | mg/kg, | mg/kg, | mg/kg, | mg/kg, |
| | male: | male: | male: | female: | female: | female: |
| | mean | mean | mean | mean | mean | mean |
| | (SD, | (SD, | (SD, | (SD, | (SD, | (SD, |
| PK parameter | CV %) | CV %) | CV %) | CV %) | CV %) | CV %) |
| T_{1/2}, h | 3.24 | 2.91 | 12.2 | 6.53 | 9.6 | 40.5 |
| | (1.26, | (0.36, | (5.5, | (0.76, | (5.3, | (40, |
| | 39) | 12.2) | 45.2) | 11.6) | 55.1) | 98.8) |
| Tₘₐₓ, h | 2.86 | 4 | 5.33 | 6 | 5.33 | 6.67 |
| | (4.46, | (2, | (1.15, | (0, | (2.31, | (1.15, |
| | 156) | 50) | 21.7) | 0) | 43.3) | 17.3) |
| Cₘₐₓ, ng/mL | 44233 | 92300 | 143667 | 68567 | 171333 | 223000 |
| | (12644, | (14722, | (26951, | (6405, | (11590, | (29866, |
| | 28.6) | 15.9) | 18.8) | 9.3) | 6.76) | 13.4) |
| AUCₗₐₛₜ, | 351930 | 905824 | 2085035 | 947420 | 2541137 | 4467175 |
| h*ng/mL | (139125, | (39934, | (193187, | (135087, | (502767, | (704885, |
| | 39.5) | 4.41) | 9.27) | 14.3) | 19.8) | 15.8) |
| AUC_{Inf}, | 356683 | 910102 | 2888539 | 1044472 | 3274996 | 16320220 |
| h*ng/mL | (140112, | (39184, | (359261, | (173985, | (1267800, | (14774911, |
| | 39.3) | 4.31) | 12.4) | 16.7) | 38.7) | 90.5) |
| AUC_{_{—}}_{% Extrap}_{_{—}}obs, | 1.28 | 0.473 | 26.5 | 9 | 18.5 | 59.9 |
| % | (1.78, | (0.225, | (16.5, | (2.5, | (15.9, | (21.2, |
| | 139) | 47.7) | 62.2) | 27.6) | 85.7) | 35.5) |
| MRT_{Inf}_{_{—}}obs, h | 6.1 | 6.03 | 18.6 | 9.9 | 14.7 | 62 |
| | (1.3, | (0.39, | (7.6, | (1.1, | (7.6, | (56, |
| | 21.3) | 6.51) | 40.8) | 11.35) | 52) | 90.4) |
| AUCₗₐₛₜ/D, | 14077 | 12078 | 8340 | 37897 | 33882 | 17869 |
| h*ng/mL | (5565, | (532, | (773, | (5403, | (6704, | (2820, |
| | 39.5) | 4.41) | 9.27) | 14.3) | 19.8) | 15.8) |

### Dog Model

Groups, Dosing, and Collection. The pharmacokinetics of the free acid and the tris salt of Compound 1 administered by oral capsules was assessed in dogs. Three male beagle dogs were administered a single dose of 3 mg/kg Compound 1 free acid on Day 1 of the study, and a single dose of 3 mg/kg Compound 1 tris salt on Day 8 of the study. Plasma was collected pre-dose, and at 0.5, 1, 2, 4, 8, 12 and 24 hours post-dose via puncture of peripheral veins. No abnormal clinical symptoms were observed.

In a follow-up study, the pharmacokinetics of tris salt Compound 1 were investigated via oral gavage of a 0.5% methylcellulose in saline formulation. Two Groups of animals were tested. In Group 1, three male beagle dogs were administered a single dose of 3 mg/kg Compound 1 tris salt. In Group 2, three male beagle dogs were administered a single dose of 30 mg/kg Compound 1 tris salt. Plasma was collected pre-dose, and at 0.5, 1, 2, 4, 8, 12 and 24 hours post-dose via puncture of peripheral veins. No abnormal clinical symptoms were observed.

LC MS-MS Analysis. Liquid Chromatography with tandem mass spectrometry was used to determine plasma concentrations of Compound 1 Free Acid and Compound 1 Tris Salt in plasma samples collected at prescribed time points. Instrumentation included YMC-Triart C₁₈, S-5 μm (50×2.1 mm) HPLC column, liquid chromatograph Shimadzu LC-30AD with communications bus module CBM-20A and Auto SIL-20AC HT; and a Triple Quad 5500 LC/MS/MS instrument. Tolbutamide was used as an internal standard. The following conditions were used:

### HPLC Conditions:

Mobile phases: A: 0.1% Formic acid in DI water; B: 0.1% Formic acid in Acetonitrile
Injection volume: 20 μl
Autosampler temp.: 4° C.
Retention time: Both free acid and salt: 1.37 min; tolbutamide 1.22 min
Gradient
Flow Rate: 600 (μL/min)

| Time (minute) | A (%) | B (%) |
|---|---|---|
| 0.50 | 90 | 10 |
| 0.80 | 10 | 90 |
| 1.80 | 10 | 90 |
| 2.00 | 90 | 10 |
| 2.50 | 90 | 10 |

### Mass Spectrometer Conditions:

Ionization mode: Negative
Monitoring mode: MRM
Capillary (volts): −4500
Gas Temperature: 550° C.
Ion transition:

| | | Dwell | | | | |
|---|---|---|---|---|---|---|
| | Transition | Time | DP | CE | EP | CXP |
| Compound | (m/z) | (ms) | (v) | (v) | (v) | (v) |
| Cpd. 1, Free | 460.3→432.1 | 100 | −134 | −37 | −10 | −14 |
| Acid/Salt | | | | | | |
| Tolbutamide | 269.0→169.9 | 100 | −75 | −22 | −10 | −1 |

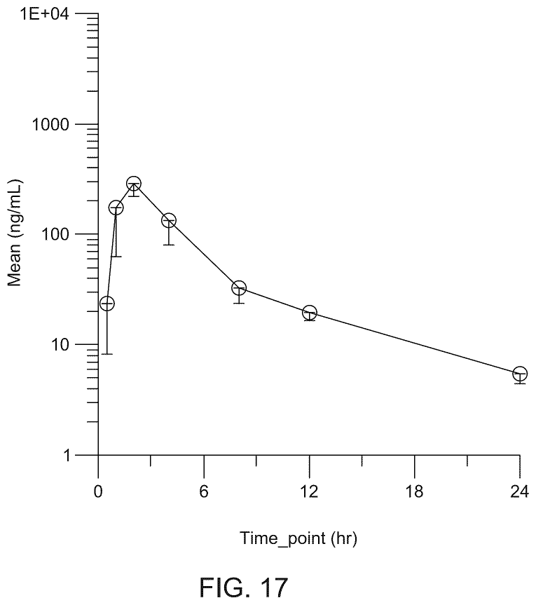

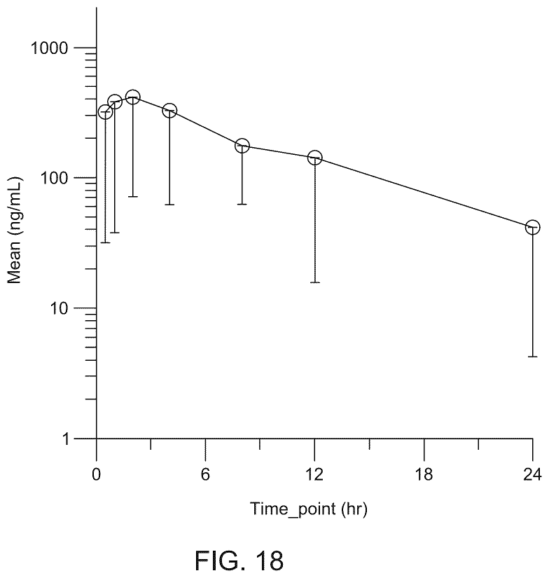

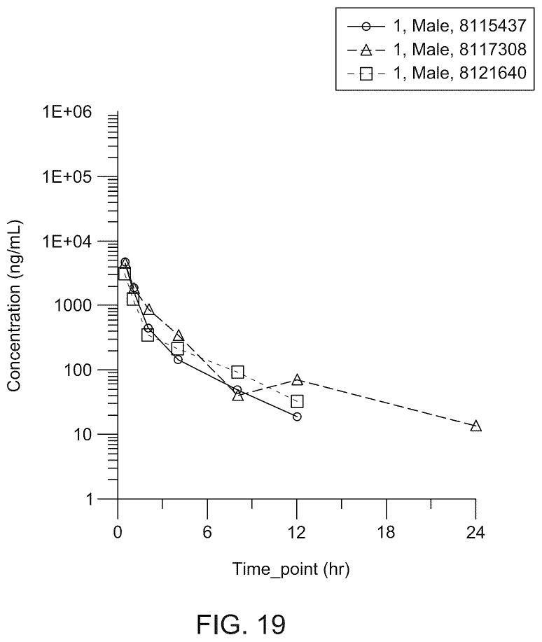

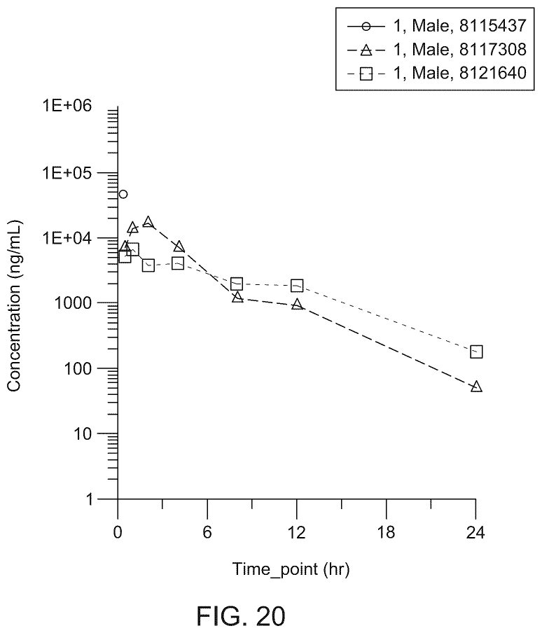

Results. Results from the powder in capsule are given below in Table 8 and FIGS. 17 and 18. Results from oral gavage of the suspension are given below at Table 9 and FIGS. 19 and 20. Overall, the total drug exposure across time (AUCₗₐₛₜ and AUC_{Inf}) was higher for the tris salt (both powder in capsule and suspension formulations) compared to the free acid. Half-life appeared lower for tris salt compared to free acid, but mean residence time increased. One animal in the salt powder in capsule experiment showed abnormally high plasma concentration at the first two timepoints (30-50× the other two subjects), so SD and CV % were not calculated.

TABLE 8
PK parameters in dogs tested with
the powder in capsule formulation.

| | Powder in Capsule | | | | | |
|---|---|---|---|---|---|---|
| | Free Acid | | | Salt | | |
| | Mean | SD | CV % | Mean | SD | CV % |
| T_{1/2} | 6.33 | 0.736 | 11.6 | 3.63 | — | — |
| AUCₗₐₛₜ | 1215 | 323 | 26.6 | 3924 | — | — |
| AUC_{inf} | 1265 | 335 | 26.5 | 4092 | — | — |
| MRTₗₐₛₜ | 5.39 | 0.796 | 14.8 | 7.89 | — | — |
| Cl_{F} | 2505 | 761 | 30.4 | 756 | — | — |

TABLE 9
PK parameters in dogs tested with the oral suspension.

| | Suspension | | | | | |
|---|---|---|---|---|---|---|
| | 3 mg/kg | | | 30 mg/kg | | |
| | Mean | SD | CV % | Mean | SD | CV % |
| T_{1/2} | 4.74 | 3.31 | 69.8 | 3.69 | — | — |
| AUCₗₐₛₜ | 5075 | 1333 | 26.3 | 431317 | 27909 | 67.5 |
| AUC_{inf} | 5202 | 1359 | 26.1 | 56792 | — | — |
| MRTₗₐₛₜ | 2.34 | 0.655 | 28.0 | 3.81 | 3.24 | 85.0 |
| Cl_{F} | 602 | 146 | 24.3 | 547 | — | — |

### Example 4: Single Crystal

Diverse solvent systems (n=4-8) were selected to grow single crystals of the compound. The solvents employed represent a range of polarities, dielectric constants, and dipole moments, and possess diverse hydrogen-bond donor/acceptor attributes. Solvents were selected based on the solubility attributes (value and temperature-dependency) and their suitability for single-crystal growth experiments.

Attempts of producing single crystals included the following crystallization techniques:
Two cooling modes using 5 solvent systems.
Slow evaporation using 5 solvent systems.
Vapor diffusion using 4 solvent combinations.
Convection mode using 1 solvent if solubility is relatively low and sufficient material is supplied.
Layering using 2 solvent systems

Polarized light microscopy, and PXRD was used to assess the crystallinity, size and quality of the solids from each crystallization experiment. PXRD was used to confirm that the desired Form 2 (Group B) was obtained.

The single-crystal growth study involved 24 experiments. Of the 24 experiments, 10 produced solids. Observed crystals were generally acicular in nature such as needles or thin blades. Most crystals were deemed too small and too thin to be suitable for SCXRD using lab based equipment. One batch of crystals from vapor diffusion (produced from DMF solution of the tris salt and acetonitrile vapor diffusing into DMF solution over a period of 7 weeks) produced larger crystals and were submitted to the crystallographer for single crystal Xray diffraction analysis. Results from the crystal growing experiments are summarized below.

### Results of Single-Crystal Growth Experiments by Slow Evaporation at Ambient Temperature

| Solvent (v:v) | Group |
|---|---|
| 2-methoxyethanol | Colorless liquid. No solids. |
| IPA:water (90:10) | Gum |
| THF:water (90:10) | Thick viscous liquid to gum |
| MeCN:water (95:5) | Yellow solution. No solids |
| methanol | Colorless liquid. No solids |

### Results of Single-Crystal Growth Experiments by Slow Cooling

| | Temperature | | |
|---|---|---|---|
| Solvent (v:v) | Range (° C.) | Group | Comments |
| 2- | 25-5 | undetermined | No solids at 5° C. Few particles |
| methoxyethanol | | | at −20° C.; no crystalline response |
| | | | by PXRD |
| IPA:water | 25-5 | B (Form 2) | Solids are Group B at 5° C. Not |
| (90:10) | | | suitable for SCXRD |
| THF:water | 25-5 | Not applicable | No solids at 5 or −20 C. ° C. |
| (90:10) | | (NA) | |
| MeCN:water | 25-5 | NA | No solids at 5° C. Few particles |
| (95:5) | | | at −20° C.; no crystalline response |
| | | | by PXRD |
| methanol | 25-5 | NA | No solids at 5° C. Few particles |
| | | | at −20° C.; no crystalline response |
| | | | by PXRD |
| 2- | 50-5 | B (Form 2) | No solids at 5° C. Few particles |
| methoxyethanol | | | at −20° C. Very weak Group B |
| | | | response by PXRD. |
| IPA:water | 50-5 | B (Form 2) | No solids at 5° C. |
| (90:10)* | | | |
| THF:water | 50-5 | NA | No solids at 5 or −20 C. ° C. |
| (90:10) | | | |
| MeCN:water | 50-5 | undetermined | No solids at 5° C. Few particles |
| (95:5) | | | at −20° C. |
| Methanol | 50-5 | undetermined | No solids at 5 C. Few particles |
| | | | at −20° C.; no crystalline response |
| | | | by PXRD |
| IPA:water | 50-5 | NA | No solids at 5 or −20° C. |
| (90:10)* | | | |

*Performed using 90:10 v/v IPA:water and slow cooling from 50 to 5° C. Solution for cooling was subsaturated with tris salt.
**Performed using 90:10 v/v IPA:water and slow cooling from 50 to 5° C. SC-011 was saturated with tris salt.

### Results of Single-Crystal Growth Experiments by Vapor Diffusion at Ambient Temperature

| Solvent | Anti-Solvent | Group | Comments |
|---|---|---|---|
| methanol | dichloromethane | NA | NA |
| chloroform | pentane | undeter- | needles too |
| | | mined | small for PXRD |
| trifluoroethanol | acetonitrile | NA | NA |
| dimethylformamide | acetonitrile | B (Form 2) | Sent for SCXRD |

### Results of Single-Crystal Growth Experiments by Convection

| | Temperature | | |
|---|---|---|---|
| Solvent | (° C.) | Group | Comments |
| ethanol | 50 | B (Form 2) | Crystals too |
| | | | small for SCXRD |

### Results of Single-Crystal Growth Experiments by Layering (Liquid-Liquid Diffusion)

| Solvent | Anti-Solvent | Temperature (° C.) | Group |
|---|---|---|---|
| Chloroform | pentane | Ambient | B (Form 2) |
| dimethylformamide | acetonitrile | Ambient | undetermined |
| dimethylformamide | acetonitrile | 5 | undetermined |

The single crystals of Form 2 (Group B) for single crystal X-ray diffraction analysis grown by vapor diffusion of acetonitrile as an anti-solvent into a solution of the tris salt in dimethylformamide were analyzed by single crystal X-ray diffraction analysis and were also used for seed crystals. Using the low temperature structure and the room temperature unit cell parameters, the simulated powder Xray diffraction pattern was calculated using Panalytical X'Pert Pro HighScore Plus, v. 2.2.0. The simulated and experimental PXRD patterns are based on copper K-alpha X-radiation as the X-ray source.

| | |
|---|---|
| Provided/Expected Formula | C27H31N3O4•C4H11NO3, Solvent: DMF/acetonitrile |
| Provided Molecular Structure | 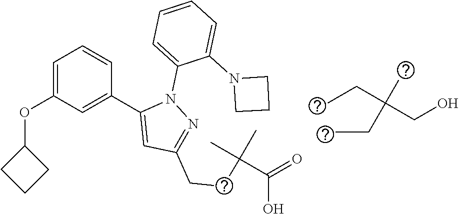 |
| Method: | Single Crystal X-ray Diffraction |
| Instrument: | Bruker D8 Venture Photon II CPAD diffractometer equipped |
| | with a CuKα INCOATEC ImuS micro-focus source (λ = |
| | 1.54178 Å). |
| Temperature, Handling | −173.15° C., Mitegen Micro Gripper, Immersion Oil NVH. |
| | Crystals were handled using oil. |
| Formula | C₄H₁₂NO₃⁺, C₂₇H₃₀N₃O₄⁻, Z = 4, No solvent is present. |
| Asymmetric unit content | C₄H₁₂NO₃⁺, C₂₇H₃₀N₃O₄⁻, Z′ = 1 |
| Symmetry | Monoclinic P2₁/c. The structure is centrosymmetric. |
| Description | The crystal is a 1:1 salt with carboxylic acid proton located at -NH3 |
| | group. Ions interact through OH. . .O and NH. . .O hydrogen bonds, |
| | including charge assisted H-bonds, to form double hydrogen bonded |
| | layer. |
| Data processing and | All non-hydrogen atoms were refined anisotropically. Hydrogen atoms |
| Refinement strategy | of —NH₃ and —OH groups were found from difference Fourier maps and |
| | freely refined. All remaining hydrogen atoms were refined using riding |
| | model. Group —OC₄H₇ is disordered over two positions and was refined |
| | with restraints. |
| Data processing | Indexing: APEX4 [1], Data integration and reduction: Saint [2]. Scaling |
| software | and Absorption correction: SADABS/TWINABS [3], Space group |
| | determination: XPREP implemented in APEX3 [1], Structure solution |
| | SHELXT [4], Refinement SHELXL-2018 [5], GUI Olex2/[6] Atomic |
| | Displacement Parameters were drawn at 50% probability. |
| Unit cell parameters at room | The unit cell at room temperature was initially |
| temperature (24.85° C. = 298K) | determined by the Difference Vectors method based on |
| | 141 reflections harvested from diffraction frames. Unit |
| | cell parameters were subsequently refined during data |
| | integration by Saint [2] and are based on 396 reflections |
| | recorded between 13.24 and 1.10Å resolution. |

| | | | | | | |
|---|---|---|---|---|---|---|
| a[Å] | b[Å] | c[Å] | α[°] | β[°] | γ[°] | V[Å³] |
| 26.526(4) | 5.940(1) | 19.055(3) | 90.000 | 90.000 | 93.123(6) | 2998(1) |

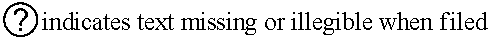

### Example 5: Summary of Safety, Toxicology, and Nonclinical Pharmacology and Pharmacokinetics

The safety pharmacology of Compound 1 and its tris salt was evaluated in an in vitro human ether-à-go-go related gene (hERG) assay, a behavioral study in Sprague Dawley rats, and a respiratory and cardiovascular system assessment using electrocardiogram (ECG)-telemetered beagle dogs. Compound 1 had a half maximal inhibitory concentration (IC₅₀) value >300 μM against the hERG channel. Determination of the IC₅₀ against the hERG channel was precluded due to potential cytotoxicity at doses >300 μM. There were no Compound 1-related behavioral effects following an oral dose of up to 400 mg/kg in male rats (the highest dose tested). There were no significant respiratory effects following an oral dose of up to 150 mg/kg in dogs (the highest dose tested). Cardiovascular safety pharmacology assessments noted decreases in PR interval duration that were detected up to 9 hours post administration of Compound 1 and were usually dose related and associated with potential changes in heart rate. These changes were considered biologically insignificant due to their small magnitude (up to 10 msec compared to vehicle control), association with potential increases in heart rate, and the absence of correlated findings.

The absorption, distribution, metabolism, and excretion properties of Compound 1 have been characterized in both in vitro and in vivo studies. Sensitive and selective bioanalytical methods have been developed and validated for rat and dog plasma. Following intravenous administration, the systemic clearance of Compound 1 in all species evaluated was considered low. Following oral administration, bioavailability was generally greater than 30% in the species evaluated. Initial studies utilized the Compound 1 free form. However, due to higher bioavailability, the tris salt form of Compound 1 was chosen for further development. See Tables 6, 7, and 10. All Good Laboratory Practice (GLP) studies were performed using the Compound 1 tris salt form. Dose levels indicate Compound 1 free form equivalents unless otherwise noted.

TABLE 10
Summary of pharmacokinetic parameters (Mean ± SD) following oral
gavage administration of Compound 1 free or tris salt form in rats.

| | | | | | | AUC₀₋₂₄ |
|---|---|---|---|---|---|---|
| Test | | | | | | salt/ |
| Article | | Route/Dose | Tₘₐₓ | Cₘₐₓ | AUC₀₋₂₄ | AUC₀₋₂₄ |
| Form | Sex | (mg/kg) | (h) | (ng/mL) | (ng h/mL) | free/ |
| Free | Male | PO/20 | 1.0 ± 0.0 | 30,600 ± 2900 | 167,000 ± 35,700 | NA |
| Free | Male | PO/60 | 2.7 ± 1.2 | 56,500 ± 8810 | 445,000 ± 129,000 | NA |
| Free | Male | PO/200 | 4.0 ± 2.0 | 115,000 ± 19,400 | 1,100,000 ± 349,000 | NA |
| Salt | Male | PO/25 | 2.9 ± 4.5 | 44,200 ± 12,600 | 352,000 ± 139,000 | 2.11 |
| Salt | Male | PO/75 | 4.0 ± 2.0 | 92,300 ± 14,700 | 906,000 ± 39,900 | 2.03 |
| Salt | Male | PO/250 | 5.3 ± 1.1 | 144,000 ± 27,000 | 2,090,000 ± 193,000 | 1.89 |
| Free | Female | PO/20 | 2.7 ± 1.1 | 78,800 ± 8460 | 885,000 ± 206,000 | NA |
| Free | Female | PO/60 | 4.7 ± 2.3 | 113,000 ± 15,700 | 1,430,000 ± 128,000 | NA |
| Free | Female | PO/200 | 4.7 ± 1.1 | 206,000 ± 26,000 | 2,850,000 ± 613,000 | NA |
| Salt | Female | PO/20 | 6.0 ± 0.0 | 68,600 ± 6400 | 947,000 ± 135,000 | 1.07 |
| Salt | Female | PO/60 | 5.3 ± 2.3 | 171,000 ± 11,600 | 2,540,000 ± 503,000 | 1.78 |
| Salt | Female | PO/200 | 6.7 ± 1.1 | 223,000 ± 29,900 | 4,470,000 ± 705,000 | 1.57 |

AUC₀₋₂₄ = Area under the concentration time curve from time zero to 24 h; Cₘₐₓ = Maximum observed concentration; NA = Not applicable; PO = Oral; SD = Standard deviation; Tₘₐₓ = Time to maximum observed concentration.

Compound 1 was administered to Sprague Dawley rats by oral gavage at doses of 25, 75, and 250 mg/kg/day (females) and 40, 120, and 400 mg/kg/day (males) for 28 days. Compound 1-related mortality occurred in 8 of the 15 toxicity males administered 400 mg/kg/day beginning on Day 8 through 24. Compound 1-related effects in males administered 400 mg/kg/day and females administered 250 mg/kg/day were decreased body weight, decreased food consumption, clinical pathology changes in hematology and serum chemistry parameters, and histopathological changes. Target tissues included bone marrow, spleen, thymus, liver, lymph nodes, epididymides, seminal vesicles, prostate, testes, uterus, and ovaries. Administration of 40 or 120 mg/kg/day Compound 1 to male and 25 or 75 mg/kg/day in female rats via oral gavage once daily (QD) for 4 weeks with a 4-week recovery phase was well tolerated. Based on these adverse findings, the no observed adverse effect level (NOAEL) was considered to be 120 mg/kg/day in male rats and 75 mg/kg/day in female rats with maximum observed concentration (Cₘₐₓ) of 180,000 ng/mL and 198,000 ng/mL, respectively, and an area under the concentration-time curve (AUC) over the time interval 0 to 24 hours post-dose (AUC₀₋₂₄) of 2,780,000 ng·h/mL and 3,230,000 ng·h/mL, respectively.

Compound 1 was administered to beagle dogs by oral gavage in a range finding study at doses of 30, 79, 240, or 790 mg/kg. Watery feces and emesis were occasionally observed in dogs at all dose levels. Male dogs administered 790 mg/kg Compound 1 had increased potassium and decreased sodium and chloride, indicating a possible Compound 1-related effect on electrolyte homeostasis. Decreased thymus weights were observed at ≥240 mg/kg and correlated with microscopic findings of decreased thymic lymphoid cellularity. Compound 1 was administered at doses of 10, 50, and 150 mg/kg/day in the pivotal repeat dose toxicity studies for 28 days. Based on a lack of adverse findings, the NOAEL was considered to be 150 mg/kg/day in dogs with an associated sex-combined Cₘₐₓ of 80,400 ng/mL and sex-combined AUC₀₋₂₄ of 474,000 ng·h/mL.

### Example 6: Effects of Compound 1 in Combination with Approved IPF Drugs in a Bleomycin-Induced Lung Fibrosis Model in Mice (Therapeutic Treatment

Compound 1 attenuates the fibroblast-to-myofibroblast transition, reducing the production of extracellular matrix protein alpha-smooth muscle actin (αSMA), in primary human lung fibroblasts. Compound 1 also reduces lung fibrosis in a bleomycin-induced mouse model of IPF, with a minimal efficacious dose of 3 mg/kg twice daily (BID). The reduction of lung fibrosis upon MCT4 inhibition is coupled with changes in serum metabolites that indicates turnover of extracellular matrix protein and increased fatty acid oxidation. Compound 1 has also demonstrated reduced lung fibrosis in the bleomycin-induced mouse model of IPF in aged (>60 week) mice, along with a reduction in lung lactate levels.

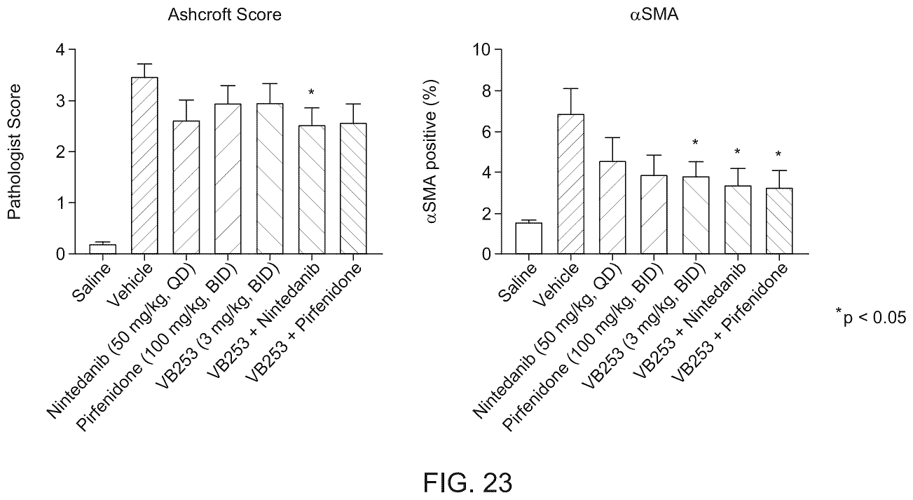

The effects of Compound 1, alone or in combination with approved IPF drugs pirfenidone or nintedanib, were studied in the bleomycin-induced lung fibrosis model in therapeutic mode. Compound 1 was dosed at 3 mg/kg, BID; pirfenidone was dosed at 100 mg/kg, BID; and nintedanib was dosed at 50 mg/kg, QD, all via oral gavage. The results of this study are shown in FIG. 23.

The addition of pirfenidone or nintedanib to Compound 1 did not produce a statistically significant additive effect upon either Ashcroft Score or αSMA quantitation.

### Example 7: Phase I Protocol

The phase 1 study will be a randomized, double-blind, placebo-controlled, single- and multiple-ascending dose study conducted in sequential groups within each study part. All Phase I studies will be performed using the Compound 1 tris salt form unless otherwise indicated. The objectives and endpoints of the study are presented in Table 11.

TABLE 11
Objectives and Endpoints

| Objectives | Endpoints |
|---|---|
| Primary: | |
| to evaluate the safety and tolerability of | incidence and severity of AEs |
| single and multiple ascending oral doses of | clinically significant changes in laboratory |
| Compound 1 tris salt and to determine a | evaluations (hematology, clinical chemistry, and |
| safe therapeutic range of Compound 1 tris | urinalysis) |
| salt in healthy subjects | clinically significant abnormal 12-lead ECG |
| to evaluate the safety and tolerability of a | parameters |
| single oral dose of Compound 1 tris salt in | clinically significant abnormal vital sign |
| healthy elderly subjects | measurements |
| to evaluate the safety and tolerability of | clinically significant abnormal physical examinations |
| multiple oral doses of Compound 1 tris salt | |
| in patients with IPF | |
| to evaluate drug safety and synergy of | |
| concomitant use of Compound 1 tris salt | |
| and approved IPF agents | |
| Secondary: | |
| to characterize the PK of Compound 1 tris | PK parameters of Compound 1 tris salt, including but |
| salt in plasma after single and multiple oral | not limited to, AUC_{0-∞}, AUC₀₋ₜₗₐₛₜ, AUC_{0-τ}, Cₘₐₓ, tₘₐₓ, |
| doses in healthy subjects, after a single dose | t_{1/2}, and AR_{AUC} in plasma and feₜ₁₋ₜ₂ in urine |
| in healthy elderly subjects, and after | comparison of the primary PK parameters of |
| multiple oral doses in patients with IPF; and | Compound 1 tris salt (AUC_{0-∞}, Cₘₐₓ [AUC₀₋ₜₗₐₛₜ may |
| in urine after a single oral dose in healthy | be included as a primary PK parameter if AUC_{0-∞} |
| subjects and elderly healthy subjects | cannot be calculated]) after single dose |
| to determine the effect of food on the PK of | administration in the fasted state and in the fed state |
| Compound 1 tris salt after a single oral dose | |
| in healthy subjects | |
| Exploratory: | |
| to assess biomarker response following | change from baseline in serum biomarkers |
| multiple oral doses of Compound 1 tris salt | (3-methylhistidine, metabolomics panel) - Parts B |
| in healthy subjects and patients with IPF | and D |
| to assess glucose uptake response following | change from baseline in glucose uptake in the lung |
| multiple oral doses of Compound 1 tris salt | via [¹⁸F]FDG-PET - Part D |
| in patients with IPF | change from baseline in FVC, FVC % pred, |
| to evaluate safety based on respiratory | FEV₁/FVC, DL_{CO}, and SpO₂ at rest - Part D |
| response in patients with IPF | Compound 1 tris salt metabolite plasma and urine |
| to identify the metabolites of Compound 1 | concentrations matrices, as available |
| tris salt in plasma and urine matrices, as | future assessment of the Compound 1 tris salt |
| available | concentration-QTc response relationship following |
| to collect ECG and PK data for the future | single oral doses of Compound 1 tris salt, spanning |
| assessment of the relationship between | potential therapeutic and supratherapeutic system |
| Compound 1 tris salt concentrations and | exposures - Part A |
| QTc following single dose administration of | |
| Compound 1 tris salt - Part A | |

Abbreviations: AE = adverse event; AR_{AUC} = accumulation ratio based on AUC_{0-τ}; AUC_{0-∞} = area under the concentration-time curve from time 0 extrapolated to infinity; AUC₀₋ₜₗₐₛₜ = area under the concentration-time curve from time 0 to the time of the last quantifiable concentration; AUC_{0-τ} = area under the concentration-time curve over a dosing interval (τ); Cₘₐₓ = maximum observed concentration; DL_{CO} = carbon monoxide diffusion capacity; ECG = electrocardiogram; [¹⁸F]FDG-PET = 2-[fluorine-18]-fluoro-2-deoxy-D-glucose positron emission tomography; feₜ₁₋ₜ₂ = percentage of the dose administered recovered over the time interval t1 to t2; FEV₁ = forced expiratory volume over 1 second; FVC = forced vital capacity; FVC % pred = percent of predicted forced vital capacity; IPF = idiopathic pulmonary fibrosis; PK = pharmacokinetics; QTc = QT interval corrected for heart rate; SpO₂ = oxygen saturation; t_{1/2} = apparent terminal elimination half-life; tₘₐₓ = time of the maximum observed concentration.

Part A will evaluate the safety, tolerability, and pharmacokinetics (PK) of a single dose of Compound 1 tris salt and food effect in healthy subjects and Part B will evaluate multiple oral doses in healthy subjects. Part C will evaluate the safety, tolerability, and PK of a single dose of Compound 1 tris salt in healthy elderly subjects. Part D will be an exploratory, multiple-dose, placebo-controlled, safety, tolerability, PK, and pharmacodynamic (PD) study conducted in patients with idiopathic pulmonary fibrosis (IPF). Parts A, B, and C will be conducted at a single site; Part D will be conducted at multiple clinical sites.

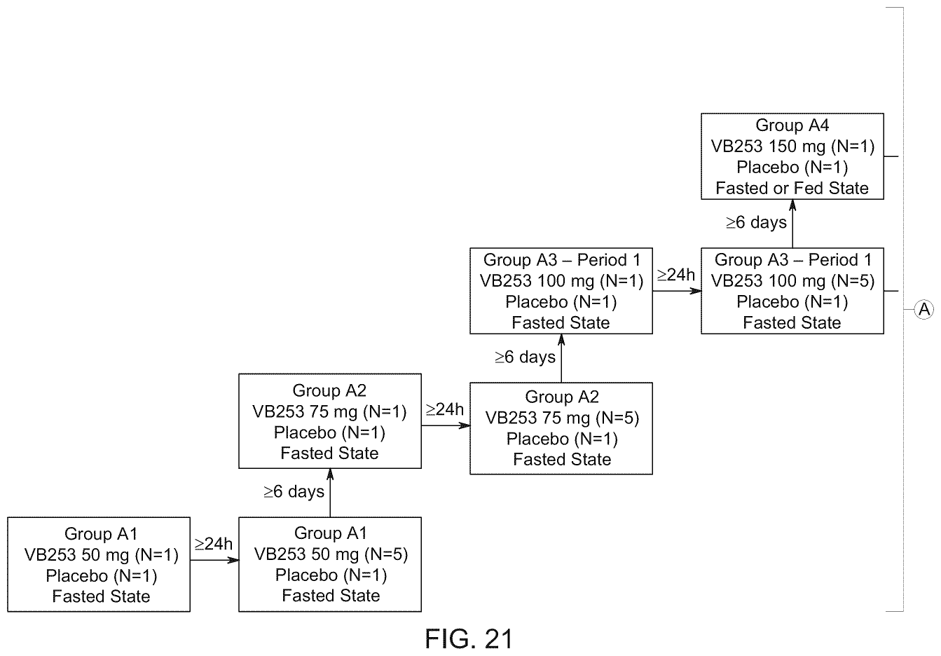

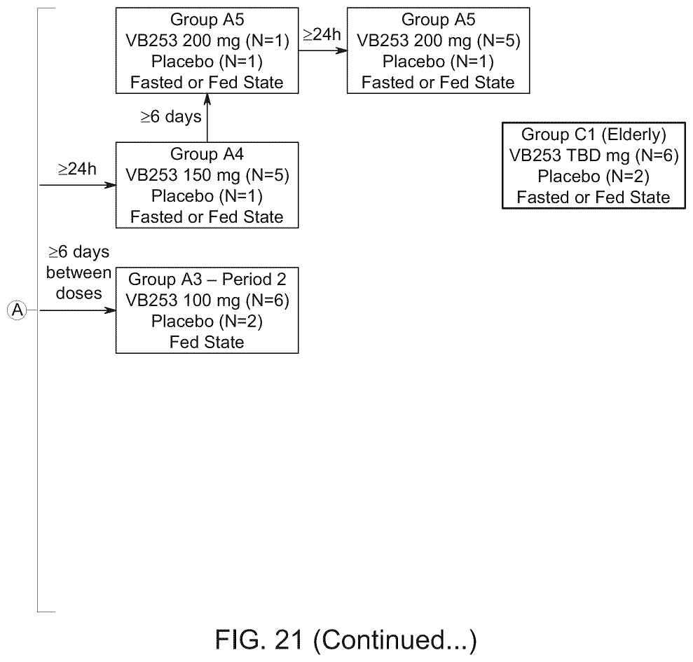

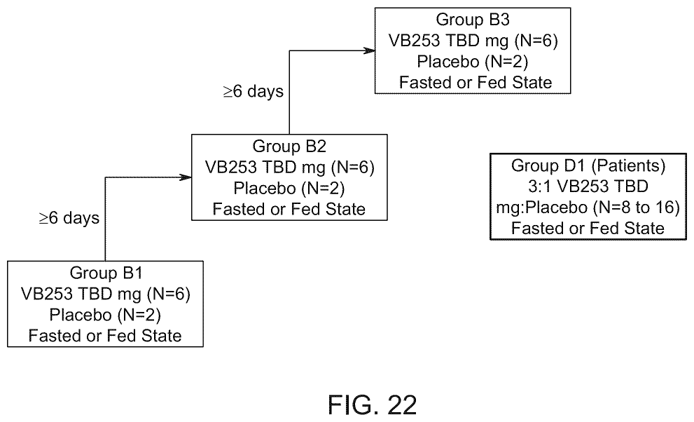

Doses may be adjusted downward or upward, delayed, not administered, or repeated. Dose increases between groups in Parts A and B will not be more than 5-fold for predicted non-pharmacologically active dose levels and will not be more than 3-fold for predicted pharmacologically active dose levels. There will be a minimum of 6 days between dose escalations to allow sufficient time for an adequate safety review. Schematics of the planned groups for Parts A and C and Parts B and D, assuming increasing dose levels for each subsequent group and sentinel dosing for each single ascending dose (SAD) group in Part A, are presented in FIGS. 21 and 22, respectively.

### Part A: Single Ascending Dose

Part A will comprise a single-dose, sequential-group design. Five dose groups of healthy subjects randomized to a single oral dose administration of Compound 1 tris salt or placebo are planned, with one group including a 2-period crossover arm to investigate the effect of food. Sentinel dosing will be used in all SAD groups. The SAD portion will include:
A screening visit from 2 days up to 4 weeks before the dose of investigational medicinal product (IMP).
An in-clinic treatment period of 4 days with check-in to the study site on Day-1, which includes a 48-hour period after the dose. Eligible subjects will be randomized on Day 1 to Compound 1 tris salt or placebo prior to administration.
A safety follow-up visit will be conducted 10 (+1) days after dose administration.

Dose administration will occur on Day 1. Safety assessments will include adverse events (AEs), laboratory evaluations (hematology, clinical chemistry, and urinalysis), electrocardiograms (ECGs), vital signs, and physical examinations. Blood and urine samples to determine the PK profile of Compound 1 tris salt will be collected from pre-dose up to 48 hours post-dose.

### Part B: Multiple Ascending Dose

Part B will comprise a multiple dose, sequential group study. Three dose groups of healthy subjects randomized to multiple oral dose administration of Compound 1 tris salt or placebo are planned. If the apparent terminal elimination half-life (t_{1/2}) of Compound 1 tris salt is found to be shorter or longer in Part A than predicted by the nonclinical data, further dose escalation may be required to include a revised dose regimen for Parts B and D. The multiple ascending dose portion will include:
A screening visit from 2 days up to 4 weeks before the first dose of IMP.
An in-clinic treatment period of 10 days with check-in to the study site on Day-1, which includes a 48-hour period after the last dose. Eligible subjects will be randomized on Day 1 to Compound 1 tris salt or placebo prior to administration.
A safety follow-up visit will be conducted 10 (+2) days after the final dose.

For all subjects, dosing is planned to be once daily (QD) on Days 1 to 7, inclusive, at approximately the same time each morning. The total daily dose administered will not exceed an exposure shown to be safe and well tolerated in Part A. Safety assessments will include AEs, laboratory evaluations (hematology, clinical chemistry, and urinalysis), ECGs, vital signs, and physical examinations. Blood samples will be collected for analysis of 3-methylhistidine and metabolomics panel prior to first dose administration on Day 1 and 4 hours post-dose on the day of final dose administration. Blood samples to determine the PK profile of Compound 1 tris salt will be collected from pre-dose up to 24 hours post-dose on Day 1; pre-dose on Days 4 through 6; and from pre-dose up to 48 hours post-dose on Day 7.

### Part C: Single Dose in Healthy Elderly Subjects

Part C will comprise a single-dose, single-group, randomized design to assess safety, tolerability, and PK in healthy elderly subjects, in order to ensure safety in elderly subjects prior to dose administration in patients with IPF. One group of healthy elderly subjects randomized to a single oral dose administration of Compound 1 tris salt or placebo is planned. The single dose evaluation will include:
A screening visit from 2 days up to 4 weeks before the dose of IMP.
An in-clinic treatment period of 4 days with check-in to the study site on Day-1, which includes a 48-hour period after the dose. Eligible subjects will be randomized on Day 1 to Compound 1 tris salt or placebo prior to administration.
A safety follow-up visit will be conducted 10 (+1) days after dose administration.

Dose administration will occur on Day 1. Safety assessments will include AEs, laboratory evaluations (hematology, clinical chemistry, and urinalysis), ECGs, vital signs, and physical examinations. Blood and urine samples to determine the PK profile of Compound 1 tris salt will be collected from pre-dose up to 48 hours post-dose.

Part D: Multiple Doses in Patients with Idiopathic Pulmonary Fibrosis

Part D will comprise a multiple-dose, single-group, randomized design to assess safety, tolerability, PK, and PD in patients with IPF. The patient portion of the study will include:
A screening visit from 2 days up to 5 weeks before the first dose of IMP.
An outpatient treatment period of a minimum of 7 days to a maximum of 28 days, which includes an outpatient visit on Day 1 followed by weekly outpatient visits. Eligible patients will be randomized on Day 1 to Compound 1 tris salt or placebo prior to administration.
A safety follow-up visit will be conducted 10 (+2) days after the final dose.

For all patients, dosing is planned to be QD at approximately the same time each morning. Doses will be administered at the study site on Day 1 and on all other outpatient visit days and will be self-administered by the patient on the remaining days. The dose level selected for Part D will be equal to or less than that assessed as safe and well tolerated in Part B. Safety assessments will include AEs, laboratory evaluations (hematology, clinical chemistry, and urinalysis), ECGs, vital signs, and physical examinations. Blood samples for PK analysis will be collected on Day 1 and on the final day of dose administration at timepoints based on the PK analysis results for Parts A, B, and C, and pre-dose on any other outpatient visit days.

Dynamic positron emission tomography imaging of the lung using the radiotracer 2 [fluorine-18]-fluoro-2-deoxy-D-glucose will be used to assess glucose uptake in the lung at screening and weekly. Blood samples will be collected for analysis of 3-methylhistidine and metabolomics panel prior to first dose administration on Day 1 and 4 hours post-dose on the day of final dose administration. Pulse oximetry for oxygen saturation (SpO₂) assessments; spirometry for forced vital capacity (FVC), forced expiratory volume over 1 second (FEV1), and FEV1/FVC assessments; and carbon monoxide diffusion capacity (DLCO) tests will be performed at screening and at outpatient visits.

### Number of Subjects

Each group in Parts A, B, and C will have 8 subjects, randomized 6:2 to Compound 1 tris salt: placebo.
For Part A, the total will be approximately 40 subjects (8 subjects×5 groups).
For Part B, the total will be approximately 24 subjects (8 subjects×3 groups).
For Part C, the total will be approximately 8 subjects (8 subjects×1 group).
For Part D, at least 8, and potentially up to 16, patients with IPF will be studied in 1 group in a ratio of approximately 3 active to 1 placebo.

Additional groups may be utilized based on the need for more evaluations or groups may be removed based on data obtained.

### Diagnosis and Main Criteria for Inclusion

Parts A and B: Healthy male and female subjects between 18 and 60 years of age, inclusive, with a body mass index between 18.0 and 32.0 kg/m², inclusive, at screening.

Part C: Healthy male and female subjects between 65 and 80 years of age, inclusive, with a body mass index between 18.0 and 32.0 kg/m², inclusive, at screening.

Part D: Male and female patients aged between 40 and 80 years (inclusive) with a diagnosis of IPF in accordance with the 2018 American Thoracic Society/European Respiratory Society/Japanese Respiratory Society/Latin American Thoracic Society Clinical Practice Guideline on Diagnosis of IPF, based on multidisciplinary team discussion and high-resolution computed tomography conducted within the previous 1 year prior to informed consent. Patients must have SpO_{2≥90}% at rest by pulse oximetry while breathing ambient air, FVC and FEV₁≥50% of predicted, ratio of FEV₁ to FVC≥0.7, and DL_{CO} corrected for hemoglobin 30% to 79% of predicted, inclusive.

### Exclusion Criteria

Parts A, B and C: Subjects in Parts A, B, and C will be excluded from the study if they satisfy any of the following criteria at the screening visit unless otherwise stated or approved by the investigator (or designee):
Significant history or clinical manifestation of any metabolic, allergic, dermatological, hepatic, renal, hematological, pulmonary, cardiovascular, gastrointestinal, neurological, respiratory, endocrine, or psychiatric disorder, history of significant hypersensitivity, intolerance, or allergy to any drug compound, food, or other substance, and/or history of stomach or intestinal surgery or resection that would potentially alter absorption and/or excretion of orally administered drugs (uncomplicated appendectomy and hernia repair will be allowed; cholecystectomy is not allowed).
Any of the following in the single 12-lead ECG at screening:
   QTcF >450 ms in males or >470 ms in females confirmed by calculating the mean of the original value and 2 repeats.
   QRS duration >110 ms confirmed by calculating the mean of the original value and 2 repeats.
   PR interval >220 ms confirmed by calculating the mean of the original value and 2 repeats.
   findings which would make QT interval corrected for heart rate (QTc) measurements difficult or QTc data uninterpretable.
   history of additional risk factors for torsades de pointes (e.g., heart failure, hypokalemia, family history of long QT syndrome).
Pulse rate ≥100 or <40 beats per minute at screening.
Positive hepatitis panel and/or positive human immunodeficiency virus test.
Administration of a coronavirus disease 2019 (COVID-19) vaccine in the past 30 days prior to dosing.
Use or intend to use any medications/products known to alter drug absorption, metabolism, or elimination processes, including St. John's wort, within 30 days prior to dosing; use or intend to use any prescription medications/products within 14 days prior to dosing; use or intend to use slow-release medications/products considered to still be active within 14 days prior to check-in; and/or use or intend to use any nonprescription medications/products including vitamins, minerals, and phytotherapeutic/herbal/plant-derived preparations within 7 days prior to check-in.
Participation in a clinical study involving administration of an investigational drug (new chemical entity) in the past 30 days or 5 half-lives (whichever is longer) prior to dosing.
Have previously completed or withdrawn from this study investigating Compound 1 and have previously received Compound 1.
Alcohol consumption of >21 units per week for males and >14 units per week for females.
Positive urine drug screen at screening or check-in or positive alcohol test result at check-in.
History of alcoholism or drug/chemical abuse within 2 years prior to check-in.
Use of tobacco- or nicotine-containing products within 3 months prior to check-in, or positive cotinine at screening or check-in.
Ingestion of poppy seed-, Seville orange-, or grapefruit-containing foods or beverages within 7 days prior to check-in.
Receipt of blood products within 2 months prior to check-in.
Donation of blood from 3 months prior to screening, plasma from 2 weeks prior to screening, or platelets from 6 weeks prior to screening.
Poor peripheral venous access.
Parts A and B: Systolic blood pressure >140 or <90 mmHg, or diastolic blood pressure >90 or <50 mmHg at screening and check in. Minor deviations from this range may be allowed if judged by the investigator to have no clinical significance.
Part C: Systolic blood pressure >150 or <90 mmHg, or diastolic blood pressure >100 or <50 mmHg at screening and check in. Minor deviations from this range may be allowed if judged by the investigator to have no clinical significance.

Part D: Patients in Part D will be excluded from the study if they satisfy any of the following criteria at the screening visit unless otherwise stated or approved by the investigator (or designee).
Serious or uncontrolled medical, surgical, or psychiatric disease that in the opinion of the investigator would compromise patient safety.
History of acute exacerbation of IPF within 3 months prior to screening, history of malignant tumor within 5 years prior to screening (with the exception of treated squamous and basal cell skin cancers and treated Stage O/in situ cervical cancer), and/or history of emphysema or clinically significant respiratory diseases (other than IPF).
Planned surgery during the study (Day 1 to follow-up visit).
Findings that are diagnostic of an alternative condition other than UIP on surgical lung biopsy (historical), HRCT imaging, transbronchial lung biopsy (historical), or bronchoalveolar lavage (historical).
Other known causes of interstitial lung disease (eg, drug toxicities, environmental exposures, connective tissue diseases).
End-stage fibrotic disease expected to require organ transplantation within 6 months.
Clinically significant findings from medical history (other than IPF), 12-lead ECG, vital sign measurements, or clinical laboratory evaluations that would compromise the safety of the patient.
Positive hepatitis panel and/or positive human immunodeficiency virus test.
Killed and inactive vaccines (e.g., pneumonia and influenza) administered ≤14 days prior to screening or live attenuated vaccines (e.g., varicella) administered ≤2 months prior to screening.
Taking a systemic corticosteroid, cytotoxic therapy (e.g., chlorambucil, azathioprine, cyclophosphamide, or methotrexate), vasodilator therapy for pulmonary hypertension (e.g., bosentan), or unapproved treatment for IPF (e.g., interferon-gamma, penicillamine, cyclosporine, mycophenolate, or N-acetylcysteine) within 4 weeks prior to screening.
   Treatment with pirfenidone or nintedanib, though not both concurrently, is permitted, provided that the patient has been on a stable dose for at least 4 weeks prior to screening and it is anticipated the dose will remain unchanged throughout enrollment (i.e., from signing the ICF to the last protocol-specified assessment, whether scheduled or unscheduled).
Use or intend to use any medications/products known to alter drug absorption, metabolism, or elimination processes, including St. John's wort, within 30 days prior to dosing; use or intend to use any new prescription medications/products within 14 days prior to dosing; use or intend to use slow-release medications/products considered to still be active within 14 days prior to Day 1 dose administration; and/or use or intend to use any nonprescription medications/products including vitamins, minerals, and phytotherapeutic/herbal/plant-derived preparations within 7 days prior to Day 1 dose administration.
Participation in a clinical study involving administration of an investigational drug (new chemical entity) in the past 30 days or 5 half-lives (whichever is longer) prior to Day 1 dose administration.
Have previously completed or withdrawn from this study investigating Compound 1 and have previously received Compound 1.
Alcohol consumption of >21 units per week for males and >14 units per week for females. One unit of alcohol equals 12 oz (360 mL) beer, 1½ oz (45 mL) liquor, or 5 oz (150 mL) wine.
Positive urine drug screen (including cotinine) or positive alcohol test result at screening or Day 1.
History of alcoholism or drug/chemical abuse within 2 years prior to Day 1 dose administration.
Use of tobacco- or nicotine-containing products within 3 months prior to Day 1 dose administration.
Ingestion of poppy seed-, Seville orange-, or grapefruit-containing foods or beverages or green, white leaf, or oolong tea or extracts within 7 days prior to Day 1 dose administration.
Receipt of blood products within 2 months prior to Day 1 dose administration.
Donation of blood from 3 months prior to screening, plasma from 2 weeks prior to screening, or platelets from 6 weeks prior to screening.
Poor peripheral venous access.

### Selection of Doses

Compound 1 tris salt was evaluated for safety in 7- and 28-day studies in rats and dogs. Compound 1 tris salt-related effects in the 28-day rat GLP study were decreased body weight, decreased food consumption, clinical pathology changes in hematology and serum chemistry parameters, and histopathological changes at 400 mg/kg/day in males and 250 mg/kg/day in females. The NOAELs for this rat study were 120 mg/kg/day for males and 75 mg/kg/day for females. There were no Compound 1 tris salt-related adverse effects in the 28-day dog GLP study. The NOAEL determined in the 28-day dog GLP study was 150 mg/kg/day. These correspond to human equivalent doses (HEDs) of:
Rat: 75 mg/kg×0.16=12 mg/kg
Dog: 150 mg/kg×0.54=81 mg/kg

Where 0.16 and 0.54 are conversion factors to extrapolate the animal dose to the HED based on body surface area.

The rat was the most sensitive species (i.e., that with the lowest HED), and assuming a 10-fold safety margin, this equates to a maximum recommended starting dose of:

$$\frac{12 mg / kg}{10} = 1.2 mg / kg$$

$$or 1.2 mg / kg \times 60 kg = 72 mg in a 60 - kg subject$$

Using the principles of allometry, the human PK parameters were predicted based on the single dose oral PK parameters. Based on the predicted human PK parameters, and the minimal effective dose in mouse IPF studies, the clinically efficacious dose was projected as approximately 30 to 80 mg.

### Investigational Medicinal Products, Dose, and Mode of Administration

25 and 100 mg Compound 1 tris salt capsules. Administration route: oral.

Proposed dose levels for Part A: Starting single dose of 50 mg, with subsequent planned doses of 75, 100, 150, and 200 mg Compound 1 tris salt. Dose increases between groups will not be more than 3-fold. Dose escalation will only occur if data from a minimum of 6 subjects have been reviewed from the previous lower dose group, such that data from a minimum of 4 subjects who have received Compound 1 tris salt will be used to make the dose escalation decision.

Proposed dose levels for Part B: The dose levels, dosing frequency, and dosing duration for Part B will be decided, in consultation with the sponsor, on the basis of data from Part A of the study. The total daily exposure of Compound 1 tris salt administered during this part of the study will not exceed an exposure shown to be safe and well tolerated in Part A. Dose escalation will only occur if data from a minimum of 6 subjects have been reviewed from the previous lower dose group, such that data from a minimum of 4 subjects who have received Compound 1 tris salt will be used to make the dose escalation decision. Dose increases between groups will not be more than 3-fold.

Proposed dose level for Part C: The dose level for Part C will be decided, in consultation with the sponsor, on the basis of data from Part A of the study. Healthy elderly subjects will be dosed at the level below the highest dose level found to be safe and well tolerated in Part A.

Proposed dose level for Part D: The dose level, dosing frequency, and dosing duration for Part D will be decided, in consultation with the sponsor, on the basis of preliminary data from Part B of the study. The total daily exposure of Compound 1 tris salt administered in Part D will not exceed an exposure shown to be safe and well tolerated in Part B. Part D may not start until review of safety and tolerability data obtained from single dose administration in healthy elderly subjects in Part C.

Dietary status for the groups in Part A prior to the food-effect evaluation group will be fasted. Dietary status for subsequent groups in Part A may be fasted or fed based on review of the preliminary PK data from the food-effect evaluation group. The dietary status for dosing in Parts B, C, and D will be determined following review of the preliminary PK data from the food effect evaluation in Part A and from earlier groups in Part B, as applicable.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

What is claimed is:

1. Form A of a compound of structural Formula I

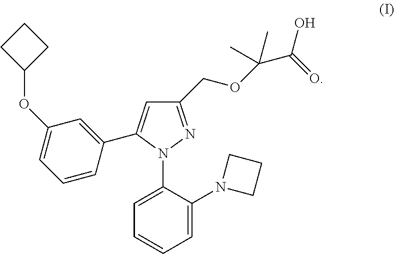 [SMILES: CC(C)(OCc1cc(-c2cccc(OC3CCC3)c2)n(-c2ccccc2N2CCC2)n1)C(=O)O]

2. The compound as recited in claim 1, which is non-solvated.

3. The compound as recited in claim 1 or 2, wherein the compound has differential scanning calorimetry data showing a primary melting endotherm with onset at about 157° C.

4. The compound as recited in claim 3, wherein the compound has a differential scanning calorimetry trace substantially as shown in FIG. 2.

5. The compound as recited in any one of claims 1 to 4, wherein the compound has a TGA trace substantially as shown in FIG. 2.

6. The compound as recited in any one of claims 1 to 5, having an FT-Raman spectrum substantially as shown in FIG. 1.

7. The compound as recited in claim 6, having an X-ray powder diffraction (XRPD) pattern substantially as shown in FIG. 3.

8. A compound of structural Formula I that is amorphous

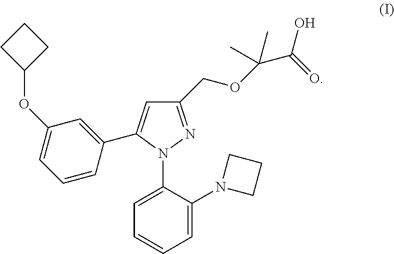 [SMILES: CC(C)(OCc1cc(-c2cccc(OC3CCC3)c2)n(-c2ccccc2N2CCC2)n1)C(=O)O]

9. Form A of a compound of structural Formula II

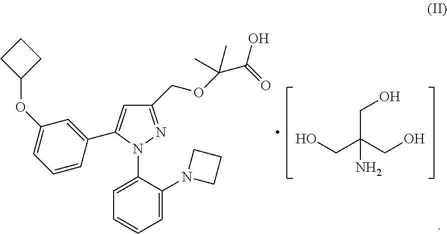

10. The compound as recited in claim 9, which is non-solvated.

11. The compound as recited in claim 9 or 10, wherein the compound has differential scanning calorimetry data showing a melting endotherm with onset at about 147° C.

12. The compound as recited in claim 11, wherein the compound has a differential scanning calorimetry trace substantially as shown in FIG. 6.

13. The compound as recited in any one of claims 9 to 12, wherein the compound has a TGA trace substantially as shown in FIG. 6.

14. The compound as recited in any one of claims 9 to 13, characterized by the presence of FT-Raman peaks of about 1604, 1438, and 995 cm⁻¹.

15. The compound as recited in claim 14, characterized by FT-Raman peaks of about 1604, 1438, 1372, 995, 332, 234, and 173 cm⁻¹.

16. The compound as recited in claim 14, having an FT-Raman spectrum substantially as shown in FIG. 5.

17. The compound as recited in any one of claims 9 to 16, having an X-ray powder diffraction (XRPD) pattern with peaks at about 7.79, 15.61, 16.71, 20.00, and 20.88±0.3 degrees two theta wherein the XRPD is measured using an incident beam of Cu radiation.

18. The compound as recited in claim 17, having an X-ray powder diffraction (XRPD) pattern with peaks at about 7.79, 12.59, 15.61, 16.71, 20.00, 20.88, and 21.50±0.3 degrees two theta wherein the XRPD is measured using an incident beam of Cu radiation.

19. The compound as recited in claim 17, having an X-ray powder diffraction (XRPD) pattern with peaks at about 7.79, 12.18, 12.59, 15.61, 16.71, 17.38, 17.72, 19.16, 20.00, 20.88, and 21.50±0.3 degrees two theta wherein the XRPD is measured using an incident beam of Cu radiation.

20. The compound as recited in claim 17, having an X-ray powder diffraction (XRPD) pattern with peaks at about 11.34, 5.67, 5.30, 4.44, and 4.25±0.3 Å in d-spacing, wherein the XRPD is measured using an incident beam of Cu radiation.

21. The compound as recited in claim 17, having an X-ray powder diffraction (XRPD) pattern with peaks at about about 11.34, 7.02, 5.67, 5.30, 4.44, 4.25 and 4.13±0.3 Å in d-spacing, wherein the XRPD is measured using an incident beam of Cu radiation.

22. The compound as recited in claim 17, having an X-ray powder diffraction (XRPD) pattern with peaks at about about 11.34, 7.26, 7.02, 5.67, 5.30, 5.10, 5.00, 4.63, 4.44, 4.25, and 4.13 Å in d-spacing, wherein the XRPD is measured using an incident beam of Cu radiation.

23. The compound as recited in claim 17, having an X-ray powder diffraction (XRPD) pattern substantially as shown in FIG. 7.

24. A process for making Form A of a compound of structural Formula II comprising combining a compound of structural Formula I with tris(hydroxymethyl)aminomethane, in a solvent and isolating Form A of the compound of structural Formula II.

25. Form B of a compound of structural Formula II

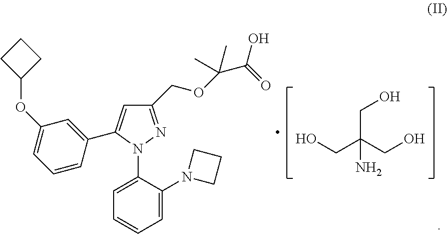

26. The compound as recited in claim 25, which is non-solvated.

27. The compound as recited in claim 25 or 26, wherein the compound has differential scanning calorimetry data showing a melting endotherm with onset at about 148° C.

28. The compound as recited in claim 27, wherein the compound has a differential scanning calorimetry trace substantially as shown in FIG. 10.

29. The compound as recited in any one of claims 25 to 28, wherein the compound has a TGA trace substantially as shown in FIG. 10.

30. The compound as recited in any one of claims 25 to 29, characterized by the presence of FT-Raman peaks of about 1601, 1545, 1468, 1437, 999, 995, and 234 cm⁻¹.

31. The compound as recited in claim 30, characterized by the presence of FT-Raman peaks of about 2946, 1601, 1545, 1507, 1468, 1437, 1374, 1345, 1043, 999, 995, 284, 234, and 186 cm⁻¹.

32. The compound as recited in any one of claims 25 to 31, having an X-ray powder diffraction (XRPD) pattern with peaks at about about 9.29, 9.70, 16.36, 19.12, and 20.15 degrees two theta wherein the XRPD is measured using an incident beam of Cu radiation.

33. The compound as recited in claim 32, having an X-ray powder diffraction (XRPD) pattern with peaks at about about 9.29, 9.70, 10.03, 16.36, 19.12, 19.49, 19.61, 20.15, and 21.68 degrees two theta wherein the XRPD is measured using an incident beam of Cu radiation.

34. The compound as recited in claim 32, having an X-ray powder diffraction (XRPD) pattern with peaks at about about 9.29, 9.70, 10.03, 11.14, 11.73, 16.36, 16.71, 19.12, 19.49, 19.61, 20.15, 20.52, 20.73, and 21.68 degrees two theta wherein the XRPD is measured using an incident beam of Cu radiation.

35. The compound as recited in claim 32, having an X-ray powder diffraction (XRPD) pattern with peaks at about about 9.51, 9.11, 5.41, 4.64, and 4.40 Å in d-spacing, wherein the XRPD is measured using an incident beam of Cu radiation.

36. The compound as recited in claim 32, having an X-ray powder diffraction (XRPD) pattern with peaks at about about 9.51, 9.11, 8.81, 5.41, 4.64, 4.55, 4.52, 4.40, and 4.10 Å in d-spacing, wherein the XRPD is measured using an incident beam of Cu radiation.

37. The compound as recited in claim 32, having an X-ray powder diffraction (XRPD) pattern with peaks at about about 9.51, 9.11, 8.81, 7.93, 7.54, 5.41, 5.30, 4.64, 4.55, 4.52, 4.40, 4.32, 4.28 and 4.10 Å in d-spacing, wherein the XRPD is measured using an incident beam of Cu radiation.

38. The compound as recited in claim 32, having an X-ray powder diffraction (XRPD) pattern substantially as shown in FIG. 11.

39. The compound as recited in claim 32, characterized by a monoclinic lattice type and P2₁/c space group having unit cell lengths for the three axes of about (a) 26.526 A, (b) 5.940 A, (c) 19.055 A and the three unit cell angles of about (a) 90.00°, (β) 90.00°, and (γ) 93.123°.

40. The compound as recited in any one of claims 25 to 39, wherein the compound is stable at 25° C. and 58% relative humidity for at least 2 weeks.

41. The compound as recited in any one of claims 25 to 39, wherein the compound is stable at 40° C. and 75% relative humidity for at least 2 weeks.

42. The compound as recited in any one of claims 25 to 39, wherein the compound is stable at 80° C. and ambient relative humidity for at least 2 weeks.

43. A process for making Form B of a compound of structural Formula II, comprising stirring Form A of a compound of structural Formula II with a suitable solvent and adding a seed crystal of Form B of a compound of structural Formula II, and isolating Form B of a compound of structural Formula II.

44. A pharmaceutical composition comprising a compound as recited in any of claims 1 to 42, and a pharmaceutically acceptable carrier, adjuvant, or vehicle.

45. A pharmaceutical composition comprising a compound as recited in any of claims 1 to 42, and a pharmaceutically acceptable carrier, adjuvant, or vehicle, wherein the composition comprises no detectable Group A tris salt.

46. A method for inhibiting activity of the monocarboxylate transporter MCT4, or a mutant thereof, in a biological sample comprising the step of contacting the biological sample with a compound as recited in any of claims 1 to 42.

47. A method for inhibiting activity of the monocarboxylate transporter MCT4, or a mutant thereof, in a patient comprising the step of administering to the patient a compound as recited in any of claims 1 to 42.

48. A method for selectively inhibiting activity of the monocarboxylate transporter MCT4, or a mutant thereof, over the monocarboxylate transporter MCT1, or a mutant thereof, in a patient comprising the step of administering to the patient a compound as recited in any of claims 1 to 42.

49. The method as recited in claim 48, wherein the inhibition is at least 100-fold selective for MCT4 over MCT1.

50. A method for treating a monocarboxylate transporter MCT4-mediated disorder in a subject in need thereof, comprising the step of administering to the subject a therapeutically effective amount of a compound as recited in any of claims 1 to 42.

51. The method as recited in claim 50, wherein the subject is a human.

52. The method as recited in claim 50, wherein the subject is in a fed state.

53. The method as recited in claim 50, wherein the subject is in a fasted state.

54. The method as recited in claim 50, wherein the monocarboxylate transporter MCT4-mediated disorder is chosen from an inflammatory disorder and a proliferative disorder.

55. The method as recited in claim 50, wherein the monocarboxylate transporter MCT4-mediated disorder is a proliferative disorder.

56. The method as recited in claim 55, wherein the proliferative disorder is cancer.

57. The method as recited in claim 56, wherein the cancer is chosen from adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, and Wilms' tumor.

58. The method as recited in claim 54, wherein the monocarboxylate transporter MCT4-mediated disorder is an inflammatory disorder.

59. The method as recited in claim 58, wherein the inflammatory disorder is chosen from Crohn's disease, ulcerative colitis, idiopathic pulmonary fibrosis, muscular dystrophy, rheumatoid arthritis, and systemic sclerosis (scleroderma).

60. The method as recited in claim 59, wherein the inflammatory disorder is idiopathic pulmonary fibrosis.

61. The method as recited in any one of claims 50 to 60, wherein the therapeutically effective amount is between about 30 mg and about 200 mg.

62. The method as recited in claim 61, wherein the therapeutically effective amount is between about 30 mg and about 80 mg.

63. The method as recited in claim 61, wherein the therapeutically effective amount is chosen from 50 mg, 75 mg, 100 mg, 150 mg, and 200 mg.

64. A method of treating a monocarboxylate transporter MCT4-mediated disorder in a subject in need thereof, comprising the sequential or co-administration of a compound as recited in any of claims 1 to 42, and another therapeutic agent.

65. The method as recited in claim 64, wherein the monocarboxylate transporter MCT4-mediated disorder is a metabolic disease.

66. The method as recited in claim 65, wherein the metabolic disease is chosen from metabolic syndrome, diabetes, dyslipidemia, fatty liver disease, non-alcoholic steatohepatitis, obesity, and insulin resistance.

67. The method of claim 66, wherein the diabetes is Type II diabetes.

68. The method of claim 66, wherein the dyslipidemia is hyperlipidemia.

69. The method of claim 64, wherein the therapeutic agent is chosen from paracetamol, acetaminophen, pirfenidone, nintedanib, and non-hormonal contraceptives.

70. A method for achieving an effect in a patient comprising the administration of a therapeutically effective amount of a compound as recited in any of claims 1 to 42 to a patient, wherein the effect is selected from the group consisting of reduction of triglycerides, reduction of cholesterol, and reduction of hemoglobin A1c.

71. The method of claim 70, wherein the cholesterol is chosen from LDL and VLDL cholesterol.

72. The method of claim 70, wherein the triglycerides are chosen from plasma triglycerides and liver triglycerides.
